(19) United States
(12) Patent Application Publication
(10) Pub. No.: US 2012/0316183 A1
(43) Pub. Date: Dec. 13, 2012

(54) NOVEL SOLVATES OF METHYLCARBAMATE

(75) Inventors: Alfons Grunenberg, Wuppertal (DE); Franz-Josef Mais, Dusseldorf (DE); Winfried Joentgen, Koln (DE); Birgit Keil, Dusseldorf (DE)

(73) Assignee: Bayer Intellecutal Property GmbH, Monheim (DE)

(21) Appl. No.: 13/515,682

(22) PCT Filed: Dec. 13, 2010
(86) PCT No.: PCT/EP10/69457

§ 371 Date: Aug. 27, 2012

(30) Foreign Application Priority Data
Dec. 14, 2009 (EP) 09179028.7

(51) Int. Cl. C07D 471/04; A61P 9/00; A61P 9/10; A61P 9/04; A61P 9/06; A61K 31/506; A61P 9/12

(52) U.S. Cl. 514/256; 544/328

(57) ABSTRACT

The invention relates to novel solvates of methyl {4,6-diamino-2-[1-(2-fluorobenzyl)-1H-pyrazolo[3,4-b]pyridin-3-yl]pyrimidin-5-yl}carbamate, in particular the semi-ethanol solvate of the formula (Ia), to processes for their preparation, to medicaments comprising them and to their use for controlling diseases

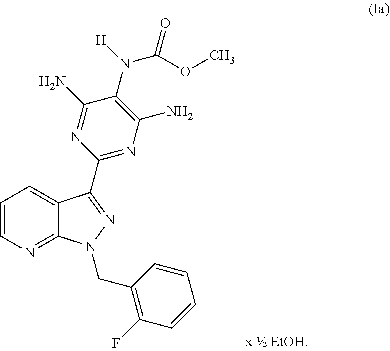

The invention relates to novel solvates of methyl {4,6-diamino-2-[1-(2-fluorobenzyl)-1H-pyrazolo[3,4-b]pyridin-3-yl]pyrimidin-5-yl}carbamate, in particular the semi-ethanol solvate of the formula (Ia), to processes for their preparation, to medicaments comprising them and to their use for controlling diseases

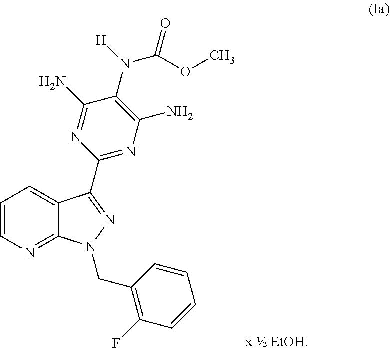

Methyl {4,6-diamino-2-[1-(2-fluorobenzyl)-1H-pyrazolo[3,4-b]pyridin-3-yl]pyrimidin-5-yl}carbamate is described in WO 03/095451 and corresponds to the compound of the formula (I)

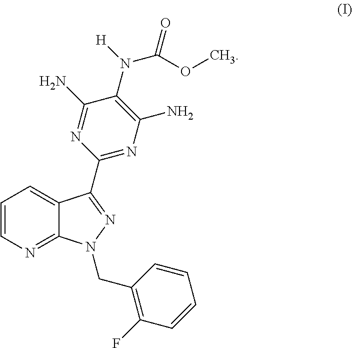

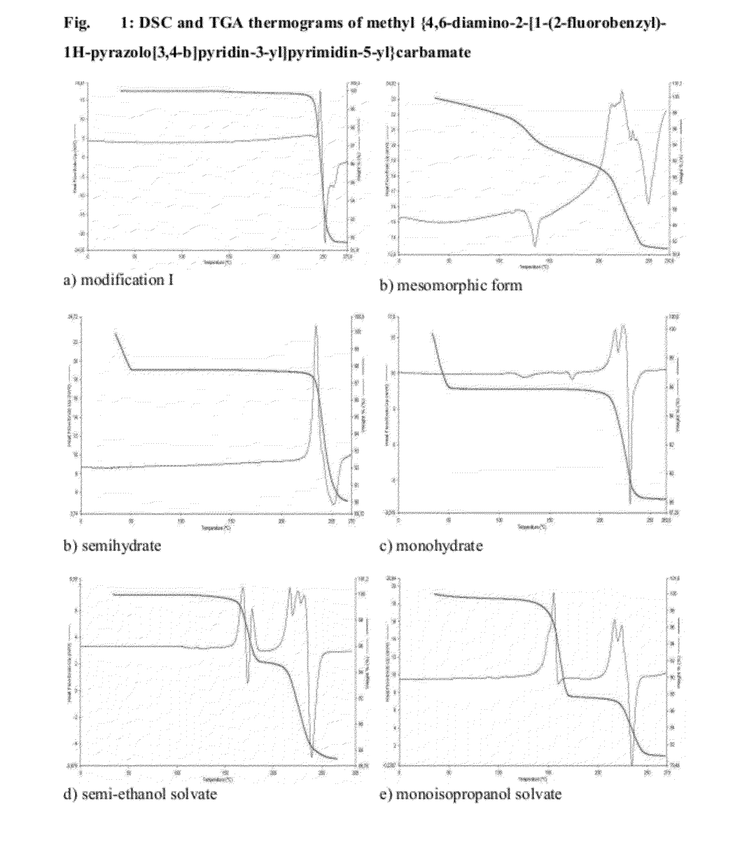

The preparation and use of the compound of the formula (I) for treating, for example cardiovascular diseases and erectile dysfunction are already known from WO 03/095451. In the manner described therein, the compound of the formula (I) is obtained in the form of a crystal modification which is referred to hereinbelow as mesomorphic form. Further polymorphic forms, in particular modification I, and the amorphous form are characterized below. The mesomorphic form has no characteristic melting point, modification I melts at 244° C. Both forms have a characteristic X-ray diffractogram, IR spectrum, Raman spectrum, FIR spectrum, NIR spectrum and ¹³C solid state NMR spectrum (Tab. 1-7, FIGS. 1-14).

It has now been found that modification I is difficult to grind and therefore has operational disadvantages in the micronization step.

Surprisingly, nine further pseuopolymorphic forms have been found. Compared to the mesomorphic form or modification I characterized in WO 03/095451 and below, the pseudo-polymorphic forms each have a characteristic X-ray diffractogram, IR spectrum, Raman spectrum, FIR spectrum, NIR spectrum and ¹³C solid state NMR spectrum (Tab. 1-7, FIGS. 1-14).

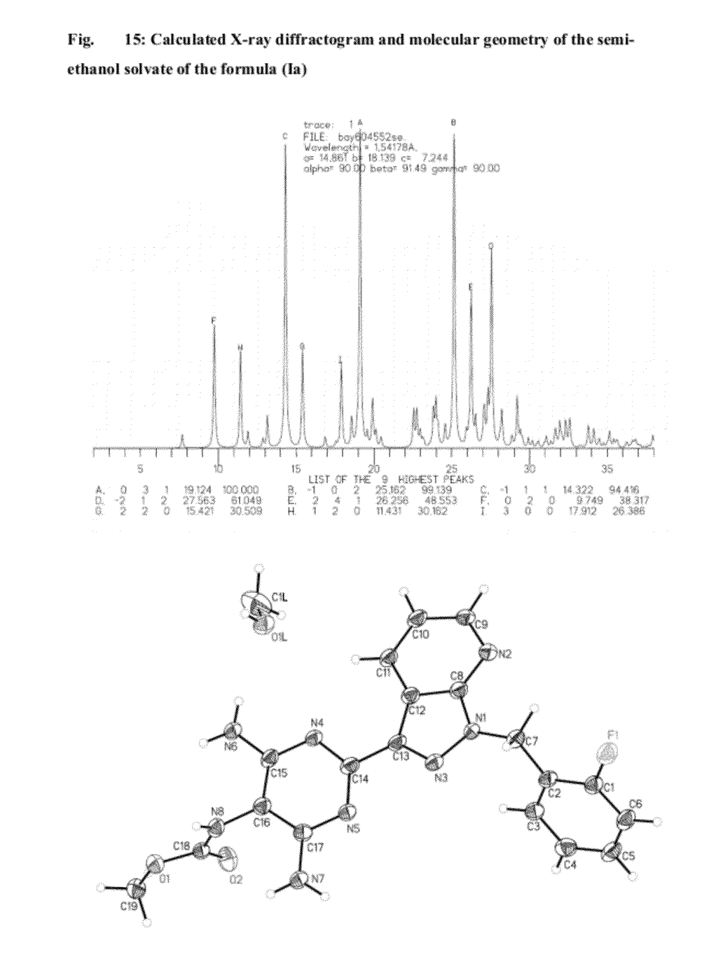

Surprisingly, a semi-ethanol solvate, a semihydrate, a monohydrate, a monoisopropanol solvate, a di-DMSO solvate, a sesquidioxane solvate, a mono-DMF solvate, a mono-NMP solvate and a THF/water form of the compound of the formula (I) have been found. The semi-ethanol solvate comprises ½ molecule of ethanol, the semihydrate ½ molecule of water, the monohydrate one molecule of water, the monoisopropanol solvate one molecule of isopropanol, the di-DMSO solvate two molecules of dimethyl sulfoxide, the sesquidioxane solvate 1.5 molecules of dioxane, the mono-DMF solvate one molecule of dimethylformamide, the mono-NMP solvate one molecule of N-methylpyrrolidone per molecule of the compound of the formula (I). The THF/water form comprises various amounts of tetrahydrofuran and water in a non-stoichiometric ratio. Compared to the mesomorphic form or modification I, characterized in WO 03/095451 and below, of the compound of the formula (I), the pseudopolymorphic forms each have a characteristic X-ray diffractogram, IR spectrum, Raman spectrum, FIR spectrum, NIR spectrum and ¹³C solid state NMR spectrum (Tab. 1-7, FIGS. 1-14). The X-ray structures of the semi-ethanol solvate, the monoisopropanol solvate, the di-DMSO solvate, the sesquidioxane solvate and the mono-DMF solvate were determined (Tab. 18, FIGS. 15-19).

The present invention provides the compound of the formula (I) as semi-ethanol solvate of the formula (Ia)

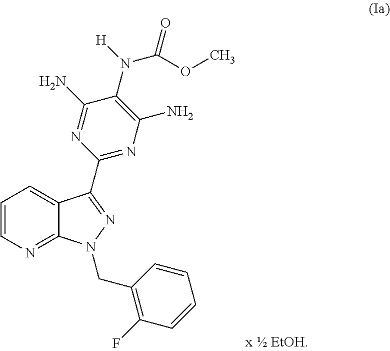

The present invention provides the compound of the formula (I) as semi-ethanol solvate of the formula (Ia), characterized in that the X-ray diffractogram of the compound has a peak maximum of the 2 theta angle at 18.8.

The present invention preferably provides the compound of the formula (I) as semi-ethanol solvate of the formula (Ia), characterized in that the X-ray diffractogram of the compound has peak maxima of the 2 theta angle at 14.0, 18.8 and 24.5.

The present invention provides the compound of the formula (I) as semi-ethanol solvate of the formula (Ia), characterized in that the NIR spectrum of the compound has peak maxima at 6851 cm⁻¹, 6017 cm⁻¹ and 4163 cm⁻¹.

The present invention furthermore provides a process for preparing the compound of the formula (Ia) by suspending the compound of the formula (Ia) for example in the mesomorphic form in an ethanol-comprising solvent and stirring or shaking at a temperature of from 10° C. to the reflux temperature of the solvent until quantitative conversion into the semi-ethanol solvate has been achieved.

General aspects in connection with the present invention are pharmacological properties, pro-cessability, preparation process, side-effect profile, stability and pharmacological activity of the semi-ethanol solvate of the formula (Ia).

Surprisingly, the semi-ethanol solvate of the formula (Ia) has, compared to modification I of the compound of the formula (I), better flowability and sievability. In addition, a higher fine-ness and a reduced tail of coarse material in the micronisate are achieved.

The compound of the formula (I) according to the invention as semi-ethanol solvate of the formula (Ia) is employed in high purity in pharmaceutical formulations. For reasons of stability, a pharmaceutical formulation comprises mainly the compound of the formula (I) as semi-ethanol solvate of the formula (Ia) and no other major fractions of any other form of the compound of the formula (I). Preferably, the medicament comprises more than 90 percent by weight, particularly preferably more than 95 percent by weight, of the compound of the formula (I) as semi-ethanol solvate of the formula (Ia), based on the total amount of the compound of the formula (I) present.

The present invention furthermore provides the use of the compound of the formula (I) as semi-ethanol solvate of the formula (Ia) for preparing a medicament for treating diseases, in particular for treating cardiovascular diseases.

The compound of the formula (I) as semi-ethanol solvate of the formula (Ia) effects a relaxation of the vessels, inhibits platelet aggregation and lowers the blood pressure, and also increases coronary blood flow. These effects are mediated via direct stimulation of soluble guanylate cyclase and an intracellular cGMP increase.

It can therefore be employed in medicaments for the treatment of cardiovascular disorders, such as, for example, for the treatment of hypertension and heart failure, stable and unstable angina pectoris, peripheral and cardiac vascular disorders, arrhythmias, for the treatment of thromboembolic disorders and ischemias, such as myocardial infarct, stroke, transitory and ischemic attacks, peripheral circulatory disorders, prevention of restenoses such as after thrombolysis therapy, percutaneous transluminal angioplasty (PTA), percutaneous transluminal coronary angioplasty (PTCA), bypass and also for the treatment of arteriosclerosis, fibrotic disorders, such as hepatic fibrosis or pulmonary fibrosis, asthmatic disorders and disorders of the urogenital system, such as, for example, prostate hypertrophy, erectile dysfunction, female sexual dysfunction and incontinence, and also for the treatment of glaucoma.

It can also be employed for controlling diseases of the central nervous system characterized by disturbances of the NO/cGMP system. In particular, it is suitable for eliminating cognitive deficits, for improving learning and memory performance and for treating Alzheimer's disease. It is also suitable for the treatment of disorders of the central nervous system, such as states of anxiety, tension and depression, sleeping disorders and sexual dysfunction caused by the central nervous system, and for regulating pathological eating disorders or disorders associated with the use of stimulants and drugs.

Furthermore, it is also suitable for regulating the cerebral circulation and is thus an effective agent for the control of migraine.

It is also suitable for the prophylaxis and control of sequelae of cerebral infarct (Apoplexia cerebri) such as stroke, cerebral ischaemias and skull-brain trauma. It can also be used for controlling states of pain.

In addition, it has an anti-inflammatory effect and can therefore be employed as an anti-inflammatory agent.

Moreover, it is suitable for treating pulmonary arterial hypertension, an impaired microcirculation, infections of the respiratory tract, reperfusion damage, respiratory disorders, lung disorders and Raynaud's syndrome.

The present invention further provides a method for treatment of disorders, in particular the disorders mentioned above, using an effective amount of the compound of the formula (I) as semi-ethanol solvate of the formula (Ia).

The compound of the formula (I) als semi-ethanol solvate of the formula (Ia) can be administered in a suitable manner, for example by the oral, parenteral, pulmonal, nasal, sublingual, lingual, buccal, rectal, dermal, transdermal, conjunctival, otic or vaginal route, or as an implant or stent.

The compound according to the invention can be administered in administration forms suitable for these administration routes.

Suitable administration forms for oral administration are those which work according to the prior art, which release the compound of the formula (I) as semi-ethanol solvate of the formula (Ia) according to the invention rapidly and/or in a modified manner, for example tablets (uncoated or coated tablets, for example with gastric juice-resistant or retarded-dissolution or insoluble coatings which control the release of the inventive compound), tablets or films/wafers which disintegrate rapidly in the oral cavity, films/lyophilizates or capsules (for example hard or soft gelatin capsules), sugar-coated tablets, granules, pellets, powders, suspensions or aerosols.

Parenteral administration can bypass an absorption step (e.g. intravenously, intraarterially, intracardially, intraspinally or intralumbally) or include an absorption (e.g. intramuscularly, subcutaneously, intracutaneously, percutaneously or intraperitoneally). Administration forms suitable for parenteral administration include preparations for injection and infusion in the form of suspensions, lyophilizates or sterile powders.

Suitable administration forms for the other administration routes are, for example, pharmaceutical forms for inhalation (including powder inhalers, nebulizers), tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), pastes, dusting powders, implants or stents.

The compound according to the invention can be converted to the administration forms mentioned. This can be done in a manner known per se, by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and flavor and/or odor correctants.

The present invention further provides medicaments which comprise at least the compound of the formula (I) as semi-ethanol solvate of the formula (Ia), typically together with one or more inert, nontoxic, pharmaceutically suitable auxiliaries such as, for example, binders, fill-ers, etc., and for the use thereof for the aforementioned purposes.

It has generally proved to be advantageous to administer the compound according to the invention in total amounts of about 0.5 to about 500, preferably 5 to 100, mg/kg of body weight per day, where appropriate in the form of a plurality of single doses, to achieve the desired results. An individual dose contains the active compound in amounts from approximately 1 to approximately 80, preferably 3 to 30, mg/kg of body weight.

The invention furthermore provides a process for preparing the compound of the formula (I) as semi-ethanol solvate of the formula (Ia) by suspending the compound of the formula (I) in any crystal form or in the amorphous form in ethanol and stirring or shaking at a temperature of from 10° C. to the reflux temperature of the solvent, preferably at from 15° C. to 35° C., particularly preferably at from 20 to 30° C., until the desired degree of conversion has been achieved, particularly preferably until quantitative conversion has been achieved. The resulting crystals of the semi-ethanol solvate are removed and the solvent present is removed by drying to constant weight at room temperature or elevated temperature.

Suitable solvents are ethanol or ethanol/water mixtures. Preference is given to ethanol.

The preparation processes are generally carried out under atmospheric pressure. However, it is also possible to operate under elevated or reduced pressure, for example at from 0.5 to 5 bar.

The percentages in the tests and examples which follow are, unless indicated otherwise, percentages by weight; parts are parts by weight. Solvent ratios, dilution ratios and concentration data for liquid/liquid solutions are based in each case on volume.

### WORKING EXAMPLES

The DSC thermograms were recorded using the differential scanning calorimeter DSC7, Pyris-1 or Diamond from Perkin-Elmer at a heating rate of 20 Kmin⁻¹. The measurements were carried out in perforated aluminum crucibles, the purge gas used was nitrogen. There was no sample preparation.

The TGA measurements were carried out using the thermal balances TGA7 and Pyris-1-TGA from Perkin-Elmer at a heating rate of 10 Kmin⁻¹. The measurements were carried out in open platinum crucibles, the purge gas used was nitrogen. There was no sample preparation.

The X-ray diffractograms were recorded at room temperature using an STOE STADI-P transmission diffractometer having a position-sensitive detector (PSD2) (radiation: copper, Kα1, primary monochromator: Ge [1 1 1], wavelength: 1.5406 Å).

The Raman spectra were recorded at room temperature using the FT-Raman spectrometers RFS 100 and Multi RAM from Bruker. The resolution is 2 cm⁻¹. There was no sample preparation. The measurement was carried out in glass tubes or on an aluminum disk.

The IR spectra were recorded at room temperature using the FT-IR spectrometers Vertex 80v and IFS 66v from Bruker. The resolution is 2 cm⁻¹. The measurement was carried out in a KBr matrix as pressed disc.

The FIR spectra were recorded at room temperature using the FT-IR spectrometers Vertex 80v and IFS 66v from Bruker. The resolution is 2 cm⁻¹. The measurement was carried out in a polyethylene matrix as pressed disc.

The NIR spectra were recorded at room temperature using a FT-NIR spectrometer IFS 28/N from Bruker. The resolution is 8 cm⁻¹. There was no sample preparation.

The solid state ¹³C NMR spectra were recorded at room temperature using a DRX 400 spectrometer from Bruker. The measurement frequency is 100.6 MHz and the rotation frequencies are 8500 Hz and 10 000 Hz. There was no sample preparation.

### Example 1

### Preparation of the semi-ethanol solvate of methyl {4,6-diamino-2-[1-(2-fluorobenzyl)-1H-Pyrazolo[3,4-b]pyridin-3-yl]pyrimidin-5-yl}carbamate of the formula (Ia)

### Example 1.1

0.1 g of methyl {4,6-diamino-2-[1-(2-fluorobenzyl)-1H-pyrazolo[3,4-b]pyridin-3-yl]pyrimidin-5-yl}carbamate in the mesomorphic form is suspended in 2 ml of ethanol, and the suspension is stirred at 50° C. After one week, the suspension is filtered and the residue is dried at room temperature and ambient humidity. The residue is examined thermoanalytically and corresponds to the title compound as semi-ethanol solvate.

### Example 1.2

3.5 l of ethanol are added to 65 g of methyl {4,6-diamino-2-[1-(2-fluorobenzyl)-1H-pyrazolo[3,4-b]pyridin-3-yl]pyrimidin-5-yl}carbamate, the substance is dissolved at reflux temperature and the solution is filtered while still hot. The filtrate is re-heated to reflux temperature, cooled and stirred at room temperature overnight. The residue is isolated, washed with ethanol and dried at 50° C. under reduced pressure. The residue is examined thermoanalytically and corresponds to the title compound as semi-ethanol solvate.

### Example 2

### Preparation of the semihydrate of methyl {4,6-diamino-2-[1-(2-fluorobenzyl)-1H-pyrazolo[3,4-b]pyridin-3-yl]pyrimidin-5-yl}carbamate

### Example 2.1

0.1 g of methyl {4,6-diamino-2-[1-(2-fluorobenzyl)-1H-pyrazolo[3,4-b]pyridin-3-yl]pyrimidin-5-yl}carbamate in modification II is suspended in 5 ml of methanol and stirred at −20° C. After 3 weeks, the suspension is filtered and the residue is dried at room temperature and ambient humidity. The residue is examined thermoanalytically and corresponds to the title compound as semihydrate.

### Example 2.2

0.1 g of methyl {4,6-diamino-2-[1-(2-fluorobenzyl)-1H-pyrazolo[3,4-b]pyridin-3-yl]pyrimidin-5-yl}carbamate in the mesomorphic form is suspended in 2 ml of methanol, and the suspension is stirred at 50° C. After one week, the suspension is filtered and the residue is dried at room temperature and ambient humidity. The residue is examined by X-ray diffractometry and corresponds to the title compound as semihydrate.

### Example 3

### Preparation of the monohydrate of methyl {4,6-diamino-2-[1-(2-fluorobenzyl)-1H-pyrazolo[3,4-b]pyridin-3-yl]pyrimidin-5-yl}carbamate

### Example 3.1

0.1 g of methyl {4,6-diamino-2-[1-(2-fluorobenzyl)-1H-pyrazolo[3,4-b]pyridin-3-yl]pyrimidin-5-yl}carbamate in the mesomorphic form is suspended in 2 ml of ethanol and shaken at 0° C. After one week, the suspension is filtered and the residue is dried at room temperature and ambient humidity. The residue is examined by X-ray diffractometry and corresponds to the title compound as monohydrate.

### Example 3.2

0.1 g of methyl {4,6-diamino-2-[1-(2-fluorobenzyl)-1H-pyrazolo[3,4-b]pyridin-3-yl]pyrimidin-5-yl}carbamate in modification II is suspended in 2 ml of methanol and stirred at room temperature. After one week, the suspension is filtered and the residue is dried at room temperature and ambient humidity. The residue is examined by X-ray diffractometry and corresponds to the title compound as monohydrate.

### Example 4

### Preparation of the monoisopropanol solvate of methyl {4,6-diamino-2-[1-(2-fluorobenzyl)-1H-pyrazolo[3,4-b]pyridin-3-yl]pyrimidin-5-yl}carbamate

### Example 4.1

0.4 g of methyl {4,6-diamino-2-[1-(2-fluorobenzyl)-1H-pyrazolo[3,4-b]pyridin-3-yl]pyrimidin-5-yl}carbamate in the mesomorphic form is dissolved in 0.6 l of hot isopropanol, and the solution is filtered. The solution is divided into three parts, and one part is allowed to stand at room temperature until the solvent has evaporated. The residue is examined thermoanalytically and corresponds to the title compound as monoisopropanol solvate.

### Example 4.2

1.5 g of methyl {4,6-diamino-2-[1-(2-fluorobenzyl)-1H-pyrazolo[3,4-b]pyridin-3-yl]pyrimidin-5-yl}carbamate in modification I are dissolved in 1.2 l of hot isopropanol, and the solution is filtered. The solution is allowed to stand in a fridge until the solvent has evaporated. The residue is examined thermoanalytically and corresponds to the title compound as monoisopropanol solvate.

### Example 4.3

80 mg of methyl {4,6-diamino-2-[1-(2-fluorobenzyl)-1H-pyrazolo[3,4-b]pyridin-3-yl]pyrimidin-5-yl}carbamate in modification I are suspended in 2 ml of isopropanol and shaken at room temperature. After one week, the suspension is filtered and the residue is dried at room temperature and ambient humidity. The residue is examined by X-ray diffractometry and corresponds to the title compound as monoisopropanol solvate.

### Example 5

### Preparation of the di-DMSO solvate of methyl {4,6-diamino-2-[1-(2-fluorobenzyl)-1H-pyrazolo[3,4-b]pyridin-3-yl]pyrimidin-5-yl}carbamate

### Example 5.1

About 10.3 kg of methyl {4,6-diamino-2-[1-(2-fluorobenzyl)-1H-pyrazolo[3,4-b]pyridin-3-yl]pyrimidin-5-yl}carbamate as isopropanol solvate are dissolved in 59.4 kg of dimethyl sulfoxide and 47.7 kg of ethyl acetate at about 90° C., and the solution is filtered. The filtrate is cooled to about 20° C. and the precipitated solid is filtered off and dried under reduced pressure at 45° C. for 24 h. The residue is examined by X-ray diffractometry and corresponds to the title compound as di-DMSO solvate.

### Example 6

### Preparation of the sesquidioxane solvate of methyl {4,6-diamino-2-[1-(2-fluorobenzyl)-1H-pyrazolo[3,4-b]pyridin-3-yl]pyrimidin-5-yl}carbamate

### Example 6.1

3.5 g of methyl {4,6-diamino-2-[1-(2-fluorobenzyl)-1H-pyrazolo[3,4-b]pyridin-3-yl]pyrimidin-5-yl}carbamate in modification I are dissolved in about 3.5 l of 1,4-dioxane and the solution is filtered and allowed to stand in a freezer for a couple of days. The solution is then allowed to stand at room temperature until the solvent has evaporated. The residue is examined by X-ray diffractometry and corresponds to the title compound as sesquidioxane solvate.

### Example 7

### Preparation of the mono-DMF solvate of methyl {4,6-diamino-2-[1-(2-fluorobenzyl)-1H-pyrazolo[3,4-b]pyridin-3-yl]pyrimidin-5-yl}carbamate

### Example 7.1

3 g of methyl {4,6-diamino-2-[1-(2-fluorobenzyl)-1H-pyrazolo[3,4-b]pyridin-3-yl]pyrimidin-5-yl}carbamate in modification I are suspended in 75 ml of dimethylformamide:water (1:1) and stirred at room temperature. After one week, the suspension is filtered and the residue is dried at room temperature and ambient humidity. The residue is exam-fined by X-ray diffractometry and corresponds to the title compound as mono-DMF solvate.

### Example 7.2

0.4 g of methyl {4,6-diamino-2-[1-(2-fluorobenzyl)-1H-pyrazolo[3,4-b]pyridin-3-yl]pyrimidin-5-yl}carbamate in modification I is dissolved in about 40 ml of dimethylformamide, and the solution is filtered. Part of the solution is allowed to stand in a fridge until the solvent has evaporated. The residue is examined by X-ray diffractometry and corresponds to the title compound as mono-DMF solvate.

### Example 8

### Preparation of the mono-NMP solvate of methyl {4,6-diamino-2-[1-(2-fluorobenzyl)-1H-pyrazolo[3,4-b]pyridin-3-yl]pyrimidin-5-yl}carbamate

### Example 8.1

3 g of methyl {4,6-diamino-2-[1-(2-fluorobenzyl)-1H-pyrazolo[3,4-b]pyridin-3-yl]pyrimidin-5-yl}carbamate in modification I are suspended in 7 ml of 1-methyl-2-pyrrolidone and stirred at room temperature. After one week, the suspension is filtered and the residue is dried at room temperature and ambient humidity. The residue is examined by X-ray diffractometry and corresponds to the title compound as mono-NMP solvate.

### Example 9

### Preparation of the THF/water form of methyl {4,6-diamino-2-[1-(2-fluorobenzyl)-1H-pyrazolo[3,4-b]pyridin-3-yl]pyrimidin-5-yl}carbamate

### Example 9.1

3 g of methyl {4,6-diamino-2-[1-(2-fluorobenzyl)-1H-pyrazolo[3,4-b]pyridin-3-yl]pyrimidin-5-yl}carbamate in modification I are dissolved in about 1 l of tetrahydrofuran, and the solution is filtered. The solution is allowed to stand at room temperature until the solvent has evaporated. The residue is examined by X-ray diffractometry and corresponds to the title compound as the THF/water form.

### Example 10

### Preparation of methyl {4,6-diamino-2-[1-(2-fluorobenzyl)-1H-pyrazolo[3,4-b]pyridin-3-yl]pyrimidin-5-yl}carbamate of the formula (I) in modification I

### Example 10.1

About 100 mg of methyl {4,6-diamino-2-[1-(2-fluorobenzyl)-1H-pyrazolo[3,4-b]pyridin-3-yl]pyrimidin-5-yl}carbamate of the formula (I) in the mesomorphic form are suspended in 3 ml of acetonitrile, and the suspension is stirred at room temperature. After one week, the suspension is filtered and the residue is dried at room temperature and ambient humidity. The residue is examined by X-ray diffractometry and corresponds to the title compound in modification I.

### Example 10.2

About 100 mg of methyl {4,6-diamino-2-[1-(2-fluorobenzyl)-1H-pyrazolo[3,4-b]pyridin-3-yl]pyrimidin-5-yl}carbamate of the formula (I) in the mesomorphic form are suspended in 2 ml of acetone, and the suspension is stirred at 50° C. under reflux. After one week, the suspension is filtered and the residue is dried at room temperature and ambient humidity.

The residue is examined by X-ray diffractometry and corresponds to the title compound in modification I.

### Example 10.3

7.1 kg of methyl {4,6-diamino-2-[1-(2-fluorobenzyl)-1H-pyrazolo[3,4-b]pyridin-3-yl]pyrimidin-5-yl}carbamate of the formula (I) as di-DMSO solvate are suspended in 171.6 kg of ethyl acetate and 42 kg of ethanol, and the suspension is stirred at about 73° C. under reflux for 20 h. The suspension is cooled to RT, filtered off with suction and washed with ethyl acetate and water. The moist product is dried at 50° C. under reduced pressure. It is examined by X-ray diffractometry and corresponds to the title compound in modification I.

### Example 11

### Preparation of methyl {4,6-diamino-2-[1-(2-fluorobenzyl)-1H-pyrazolo[3,4-b]pyridin-3-yl]pyrimidin-5-yl}carbamate of the formula (I) in modification II

### Example 11.1

110.5 g of methyl {4,6-diamino-2-[1-(2-fluorobenzyl)-1H-pyrazolo[3,4-b]pyridin-3-yl]pyrimidin-5-yl}carbamate of the formula (I) as HCl salt are suspended in 1960 ml of ethanol at room temperature. 140 ml of triethylamine are metered in, and the mixture is stirred at RT for another 3 h. The solid is filtered off with suction and washed with ethanol. The moist product is dried at 50° C. under reduced pressure overnight. It is examined by X-ray diffractometry and corresponds to the title compound in modification II.

TABLE 1
Thermogravimetry

| | Mass loss |
|---|---|
| | [% by weight] |
| modification I | <0.5 |
| mesomorphic | about 8 |
| semi-ethanol solvate | 5.3 |
| monohydrate | 4.2 |
| semihydrate | 2.2 |
| monoisopropanol solvate | 12.8 |
| di-DMSO solvate | 27.7 |
| sesquidioxane solvate | 24.4 |
| mono-DMF solvate | 15.2 |
| mono-NMP solvate | 19.5 |
| THF/water form | about 9 |
| modification II | <0.5 |

TABLE 2
X-Ray diffractometry
Peak maximum [2 theta]

| | | | | semi- | monoiso- | |
|---|---|---|---|---|---|---|
| modifi- | meso- | semi- | mono- | ethanol | propanol | di-DMSO |
| cation I | morphic | hydrate | hydrate | solvate | solvate | solvate |
| 3.6 | 4.0 | 3.4 | 6.7 | 9.6 | 5.1 | 6.8 |
| 4.9 | 5.2 | 4.9 | 8.5 | 11.3 | 8.3 | 8.3 |
| 6.1 | 6.1 | 5.9 | 13.8 | 11.9 | 8.9 | 8.8 |
| 7.0 | 9.1 | 6.2 | 14.1 | 12.8 | 9.1 | 9.2 |
| 7.3 | 13.0 | 8.8 | 14.5 | 14.0 | 9.8 | 9.7 |
| 8.8 | 15.2 | 9.9 | 16.7 | 15.3 | 11.8 | 10.1 |
| 9.9 | 16.9 | 10.4 | 18.0 | 16.5 | 12.3 | 11.1 |
| 10.9 | 17.5 | 11.2 | 18.2 | 17.9 | 13.0 | 11.4 |
| 12.0 | 21.0 | 11.8 | 18.5 | 18.8 | 13.5 | 11.6 |
| 12.3 | 23.7 | 12.4 | 18.7 | 19.3 | 14.4 | 11.9 |
| 14.7 | 25.6 | 13.9 | 19.4 | 19.6 | 15.4 | 12.1 |
| 15.3 | | 14.4 | 20.3 | 19.8 | 16.2 | 12.4 |
| 16.5 | | 14.9 | 21.5 | 20.3 | 16.8 | 12.9 |
| 17.6 | | 15.1 | 21.8 | 22.4 | 16.9 | 13.5 |
| 18.2 | | 15.5 | 23.5 | 22.7 | 17.4 | 13.7 |
| 18.4 | | 17.4 | 24.2 | 23.6 | 18.3 | 13.8 |
| 19.8 | | 17.7 | 24.6 | 23.8 | 19.4 | 14.2 |
| 20.8 | | 18.8 | 25.3 | 24.5 | 19.8 | 14.4 |
| 21.1 | | 19.5 | 25.6 | 25.2 | 20.3 | 15.0 |
| 21.3 | | 20.7 | 26.3 | 25.9 | 20.9 | 15.6 |
| 21.8 | | 21.1 | 26.7 | 26.4 | 21.3 | 16.2 |
| 22.2 | | 21.2 | 27.2 | 26.6 | 21.6 | 16.4 |
| 22.9 | | 21.6 | 28.4 | 27.0 | 22.2 | 16.5 |
| 24.1 | | 22.0 | 30.2 | 27.4 | 22.7 | 16.8 |
| 24.4 | | 22.5 | 31.7 | 27.8 | 23.0 | 16.9 |
| 24.7 | | 23.4 | 33.3 | 28.3 | 23.2 | 17.1 |
| 25.6 | | 24.0 | 35.8 | 28.7 | 23.7 | 17.5 |
| 26.0 | | 24.3 | 36.7 | 29.0 | 24.3 | 17.9 |
| 26.8 | | 24.7 | | 29.2 | 24.7 | 18.0 |
| 27.4 | | 25.1 | | 29.6 | 25.2 | 18.4 |
| 27.8 | | 25.7 | | 30.4 | 25.3 | 18.7 |
| 28.1 | | 26.5 | | 30.9 | 25.5 | 19.0 |
| 28.3 | | 26.8 | | 31.6 | 25.8 | 19.4 |
| 29.3 | | 27.3 | | 32.2 | 26.1 | 19.6 |
| 29.7 | | 27.5 | | 33.4 | 26.3 | 20.3 |
| 30.1 | | 28.0 | | 34.8 | 26.6 | 20.6 |
| 30.9 | | 28.2 | | 36.3 | 27.2 | 20.8 |
| 31.7 | | 28.7 | | 36.9 | 27.6 | 21.2 |
| 32.0 | | 29.1 | | 37.5 | 27.9 | 21.8 |
| 32.7 | | 29.5 | | 37.9 | 28.2 | 22.2 |
| 33.0 | | 29.8 | | | 28.5 | 22.4 |
| 33.5 | | 30.3 | | | 29.2 | 22.6 |
| 34.2 | | 30.5 | | | 29.6 | 22.9 |
| 35.3 | | 31.0 | | | 29.9 | 23.2 |
| 35.6 | | 32.8 | | | 30.2 | 23.3 |
| 36.0 | | 33.2 | | | 30.6 | 23.6 |
| | | 33.6 | | | 30.9 | 24.0 |
| | | 34.0 | | | 31.9 | 24.3 |
| | | 35.6 | | | 32.5 | 24.6 |
| | | 35.9 | | | 32.6 | 25.1 |
| | | | | | 32.9 | 25.4 |
| | | | | | 33.9 | 25.8 |
| | | | | | 34.3 | 26.1 |
| | | | | | 34.6 | 26.5 |
| | | | | | 34.9 | 26.9 |
| | | | | | 35.1 | 27.6 |
| | | | | | 35.3 | 28.5 |
| | | | | | 35.6 | 28.8 |
| | | | | | 36.0 | 29.0 |
| | | | | | 36.8 | 29.8 |
| | | | | | 37.4 | 30.0 |
| | | | | | | 30.2 |
| | | | | | | 30.6 |

| sesqui- | mono- | mono- | THF/ | |
|---|---|---|---|---|
| dioxane | DMF | NMP | water | modification |
| solvate | solvate | solvate | form | II |
| 7.9 | 8.2 | 7.5 | 5.8 | 8.3 |
| 8.5 | 9.2 | 8.6 | 6.1 | 11.3 |
| 9.2 | 9.7 | 9.3 | 8.3 | 11.5 |
| 11.5 | 11.9 | 9.9 | 9.1 | 12.1 |
| 12.5 | 12.5 | 11.4 | 9.3 | 13.6 |
| 13.7 | 12.8 | 11.7 | 9.8 | 14.1 |
| 14.6 | 13.3 | 12.2 | 11.9 | 14.8 |
| 14.8 | 14.2 | 12.7 | 12.4 | 16.3 |
| 15.1 | 15.6 | 13.5 | 13.0 | 17.0 |
| 15.8 | 16.0 | 14.9 | 13.4 | 17.5 |
| 16.1 | 16.5 | 15.3 | 14.4 | 18.2 |
| 16.4 | 16.8 | 16.1 | 14.7 | 19.0 |
| 16.9 | 17.6 | 17.0 | 15.1 | 21.1 |
| 17.1 | 18.3 | 17.2 | 15.3 | 22.1 |
| 17.5 | 18.6 | 17.4 | 15.6 | 22.9 |
| 17.6 | 19.4 | 17.8 | 16.1 | 23.3 |
| 18.0 | 19.8 | 18.0 | 16.6 | 24.0 |
| 18.3 | 20.0 | 18.4 | 16.8 | 25.1 |
| 18.5 | 20.5 | 18.7 | 17.5 | 25.4 |
| 18.6 | 20.6 | 19.0 | 18.3 | 26.1 |
| 19.4 | 21.0 | 19.2 | 18.7 | 26.7 |
| 19.7 | 21.3 | 19.4 | 19.4 | 28.6 |
| 20.5 | 22.0 | 19.8 | 19.7 | 29.3 |
| 20.6 | 22.4 | 20.1 | 20.8 | 30.4 |
| 21.5 | 22.6 | 20.2 | 21.1 | 34.0 |
| 22.0 | 22.9 | 20.4 | 21.4 | 35.6 |
| 22.1 | 23.2 | 22.2 | 22.4 | 36.9 |
| 22.3 | 23.6 | 22.5 | 22.7 | 37.7 |
| 22.6 | 24.0 | 23.2 | 22.9 | 25.4 |
| 23.0 | 24.5 | 23.5 | 23.1 | 26.1 |
| 23.1 | 25.0 | 23.8 | 23.7 | 26.7 |
| 23.4 | 25.3 | 24.6 | 24.0 | 28.6 |
| 23.8 | 25.7 | 24.8 | 24.2 | 29.3 |
| 23.9 | 25.9 | 24.9 | 25.0 | 30.4 |
| 24.2 | 26.3 | 25.2 | 25.5 | 34.0 |
| 24.5 | 26.9 | 25.5 | 25.9 | 35.6 |
| 25.1 | 27.3 | 25.9 | 26.2 | 36.9 |
| 25.2 | 27.7 | 26.2 | 26.5 | 37.7 |
| 25.5 | 28.0 | 26.5 | 27.0 | |
| 25.8 | 28.3 | 27.1 | 27.4 | |
| 26.3 | 28.5 | 27.5 | 28.2 | |
| 26.9 | 28.8 | 28.2 | 28.6 | |
| 27.5 | 29.4 | 28.6 | 29.2 | |
| 28.0 | 29.7 | 28.8 | 29.5 | |
| 28.4 | 30.4 | 29.2 | 29.8 | |
| 28.8 | 30.8 | 29.6 | 30.0 | |
| 29.0 | 31.2 | 30.0 | 30.5 | |
| 29.4 | 31.6 | 30.8 | 31.3 | |
| 29.6 | 31.7 | 31.2 | 31.7 | |
| 30.0 | 32.0 | 31.7 | 32.1 | |
| 30.1 | 32.5 | 32.0 | 32.3 | |
| 30.4 | 32.7 | 32.6 | 32.7 | |
| 30.6 | 32.9 | 33.2 | 32.8 | |
| 31.4 | 33.3 | 33.4 | 34.3 | |
| 31.9 | 34.1 | 33.8 | 34.6 | |
| 32.2 | 34.5 | 34.3 | 34.9 | |
| 32.4 | 34.8 | 34.9 | 35.7 | |
| 32.7 | 35.2 | 35.3 | 36.1 | |
| 33.0 | 35.6 | 36.0 | 36.9 | |
| 33.6 | 35.8 | 36.5 | | |
| 34.1 | 36.4 | 37.7 | | |
| 34.3 | 36.8 | | | |
| 34.6 | 37.0 | | | |
| 34.9 | 37.5 | | | |
| 35.8 | | | | |
| 36.1 | | | | |
| 36.5 | | | | |
| 37.2 | | | | |
| 37.3 | | | | |
| 37.9 | | | | |

TABLE 3
IR spectroscopy
Wave number [cm⁻¹]

| | | | | semi- | monoiso- | di- |
|---|---|---|---|---|---|---|
| modification | meso- | semi- | mono- | ethanol | propanol | DMSO |
| I | morphic | hydrate | hydrate | solvate | solvate | solvate |
| 3483 | 3633 | 3656 | 3642 | 3488 | 3492 | 3421 |
| 3470 | 3443 | 3454 | 3502 | 3464 | 3429 | 3325 |
| 3451 | 3330 | 3388 | 3458 | 3379 | 3275 | 3225 |
| 3387 | 3222 | 3332 | 3356 | 3333 | 3126 | 3156 |
| 3330 | 2952 | 3211 | 3280 | 3274 | 2979 | 2996 |
| 3276 | 1705 | 3147 | 3209 | 3202 | 2966 | 2950 |
| 3214 | 1630 | 2953 | 3104 | 3146 | 1893 | 1722 |
| 3133 | 1566 | 1707 | 2955 | 2959 | 1711 | 1632 |
| 2952 | 1511 | 1631 | 1703 | 2916 | 1626 | 1568 |
| 1712 | 1492 | 1568 | 1622 | 1695 | 1599 | 1517 |
| 1636 | 1477 | 1509 | 1563 | 1619 | 1565 | 1481 |
| 1567 | 1437 | 1479 | 1492 | 1587 | 1512 | 1457 |
| 1509 | 1390 | 1442 | 1480 | 1565 | 1492 | 1439 |
| 1478 | 1351 | 1387 | 1456 | 1492 | 1482 | 1400 |
| 1441 | 1323 | 1351 | 1439 | 1481 | 1439 | 1364 |
| 1387 | 1288 | 1323 | 1389 | 1458 | 1381 | 1324 |
| 1350 | 1277 | 1305 | 1351 | 1435 | 1355 | 1293 |
| 1323 | 1247 | 1289 | 1320 | 1422 | 1325 | 1275 |
| 1289 | 1232 | 1276 | 1287 | 1390 | 1305 | 1237 |
| 1276 | 1174 | 1250 | 1275 | 1364 | 1287 | 1186 |
| 1249 | 1140 | 1233 | 1232 | 1324 | 1274 | 1172 |
| 1232 | 1112 | 1173 | 1174 | 1277 | 1261 | 1143 |
| 1174 | 1061 | 1139 | 1141 | 1267 | 1231 | 1108 |
| 1139 | 1030 | 1113 | 1075 | 1259 | 1193 | 1075 |
| 1111 | 940 | 1077 | 1033 | 1230 | 1171 | 1043 |
| 1086 | 911 | 1062 | 939 | 1193 | 1140 | 1020 |
| 1075 | 863 | 1031 | 912 | 1168 | 1122 | 953 |
| 1062 | 846 | 941 | 897 | 1143 | 1086 | 912 |
| 1031 | 820 | 911 | 841 | 1096 | 1072 | 840 |
| 1004 | 808 | 868 | 821 | 1062 | 1055 | 810 |
| 941 | 797 | 850 | 808 | 941 | 1028 | 779 |
| 911 | 774 | 820 | 773 | 913 | 965 | 765 |
| 867 | 757 | 809 | 763 | 882 | 947 | 715 |
| 849 | 712 | 795 | 631 | 841 | 909 | 668 |
| 820 | 643 | 774 | 589 | 823 | 847 | 628 |
| 808 | 621 | 758 | 573 | 809 | 824 | 557 |
| 795 | 590 | 714 | 513 | 796 | 816 | 514 |
| 774 | 568 | 631 | | 774 | 809 | |
| 760 | 534 | 592 | | 767 | 802 | |
| 715 | 519 | 577 | | 703 | 779 | |
| 631 | | 535 | | 663 | 767 | |
| 594 | | | | 589 | 685 | |
| 576 | | | | 576 | 634 | |
| 535 | | | | 562 | 617 | |
| | | | | 547 | 594 | |
| | | | | 510 | 574 | |
| | | | | | 560 | |
| | | | | | 536 | |
| | | | | | 521 | |

| sesqui- | mono- | mono- | THF/ | |
|---|---|---|---|---|
| dioxane | DMF | NMP | water | modification |
| solvate | solvate | solvate | form | II |
| 3433 | 3492 | 3498 | 3490 | 3507 |
| 3113 | 3417 | 3339 | 3452 | 3484 |
| 3004 | 3242 | 3271 | 3386 | 3397 |
| 2933 | 3151 | 3214 | 3327 | 3291 |
| 2852 | 3027 | 3094 | 3278 | 3158 |
| 1963 | 1954 | 2949 | 3146 | 3024 |
| 1635 | 1931 | 2871 | 2953 | 2955 |
| 1609 | 1728 | 1725 | 1710 | 1724 |
| 1583 | 1658 | 1657 | 1626 | 1632 |
| 1530 | 1642 | 1625 | 1564 | 1608 |
| 1498 | 1620 | 1586 | 1510 | 1562 |
| 1466 | 1563 | 1566 | 1478 | 1491 |
| 1445 | 1512 | 1526 | 1439 | 1477 |
| 1412 | 1491 | 1497 | 1391 | 1437 |
| 1383 | 1480 | 1489 | 1353 | 1386 |
| 1362 | 1451 | 1480 | 1323 | 1345 |
| 1325 | 1437 | 1454 | 1289 | 1322 |
| 1306 | 1412 | 1439 | 1276 | 1287 |
| 1279 | 1388 | 1422 | 1249 | 1275 |
| 1240 | 1356 | 1408 | 1232 | 1235 |
| 1161 | 1325 | 1389 | 1174 | 1170 |
| 1136 | 1307 | 1370 | 1140 | 1141 |
| 1094 | 1275 | 1359 | 1112 | 1112 |
| 1060 | 1244 | 1325 | 1058 | 1087 |
| 1024 | 1230 | 1303 | 1030 | 1071 |
| 980 | 1195 | 1289 | 941 | 1030 |
| 952 | 1179 | 1263 | 910 | 995 |
| 917 | 1140 | 1243 | 867 | 937 |
| 898 | 1114 | 1223 | 848 | 907 |
| 825 | 1095 | 1182 | 821 | 874 |
| 795 | 1088 | 1169 | 809 | 849 |
| 755 | 1073 | 1148 | 774 | 812 |
| 715 | 1056 | 1110 | 759 | 799 |
| 659 | 1027 | 1094 | 531 | 781 |
| 636 | 1001 | 1077 | 593 | 665 |
| 623 | 962 | 1033 | 574 | 671 |
| | 942 | 986 | 535 | 644 |
| | 909 | 942 | | 627 |
| | 869 | 913 | | 587 |
| | 847 | 862 | | 570 |
| | 826 | 838 | | 536 |
| | 809 | 824 | | |
| | 791 | 812 | | |
| | 777 | 798 | | |
| | 771 | 783 | | |
| | 745 | 767 | | |
| | 728 | 719 | | |
| | 715 | 645 | | |
| | 664 | 585 | | |
| | 637 | 571 | | |
| | 593 | 512 | | |
| | 571 | | | |
| | 560 | | | |
| | 537 | | | |
| | 523 | | | |

TABLE 4
Raman spectroscopy
Wave number [cm⁻¹]

| | | | | semi- | monoiso- | di- |
|---|---|---|---|---|---|---|
| modification | meso- | semi- | mono- | ethanol | propanol | DMSO |
| I | morphic | hydrate | hydrate | solvate | solvate | solvate |
| 3452 | 3067 | 3082 | 3074 | 3092 | 3069 | 3061 |
| 3387 | 3024 | 3054 | 2959 | 3069 | 3058 | 2998 |
| 3331 | 2956 | 3022 | 2937 | 3039 | 3026 | 2950 |
| 3086 | 2608 | 2954 | 2587 | 3011 | 2981 | 2916 |
| 3054 | 1704 | 2609 | 1638 | 2962 | 2955 | 2835 |
| 3022 | 1618 | 1704 | 1615 | 2918 | 2936 | 1731 |
| 2990 | 1578 | 1617 | 1601 | 2833 | 1709 | 1642 |
| 2953 | 1508 | 1576 | 1570 | 2603 | 1649 | 1615 |
| 2834 | 1479 | 1507 | 1506 | 1692 | 1631 | 1594 |
| 2604 | 1448 | 1447 | 1485 | 1617 | 1616 | 1567 |
| 1702 | 1423 | 1420 | 1446 | 1599 | 1600 | 1505 |
| 1633 | 1380 | 1380 | 1424 | 1577 | 1575 | 1485 |
| 1618 | 1323 | 1351 | 1384 | 1508 | 1506 | 1446 |
| 1598 | 1309 | 1322 | 1371 | 1497 | 1487 | 1420 |
| 1577 | 1278 | 1306 | 1318 | 1481 | 1442 | 1391 |
| 1508 | 1252 | 1276 | 1298 | 1447 | 1423 | 1373 |
| 1477 | 1233 | 1249 | 1230 | 1422 | 1390 | 1323 |
| 1447 | 1177 | 1227 | 1175 | 1388 | 1373 | 1308 |
| 1420 | 1157 | 1174 | 1156 | 1376 | 1355 | 1254 |
| 1380 | 1142 | 1141 | 1143 | 1363 | 1323 | 1231 |
| 1351 | 1114 | 1113 | 1066 | 1324 | 1307 | 1186 |
| 1322 | 1063 | 1063 | 1033 | 1279 | 1275 | 1174 |
| 1307 | 1036 | 1034 | 925 | 1259 | 1248 | 1154 |
| 1289 | 964 | 960 | 820 | 1228 | 1230 | 1145 |
| 1277 | 823 | 911 | 806 | 1192 | 1178 | 1110 |
| 1249 | 798 | 822 | 797 | 1170 | 1153 | 1027 |
| 1225 | 777 | 795 | 775 | 1157 | 1142 | 959 |
| 1175 | 742 | 776 | 764 | 1145 | 1115 | 910 |
| 1157 | 717 | 741 | 741 | 1097 | 1056 | 825 |
| 1140 | 645 | 716 | 717 | 1063 | 1034 | 798 |
| 1112 | 591 | 645 | 627 | 1037 | 1003 | 771 |
| 1064 | 560 | 591 | 609 | 1003 | 965 | 743 |
| 1034 | 536 | 559 | 566 | 963 | 908 | 716 |
| 961 | 521 | 534 | 539 | 912 | 823 | 681 |
| 910 | 472 | 446 | 515 | 883 | 816 | 670 |
| 823 | 447 | 364 | 465 | 842 | 788 | 633 |
| 808 | 408 | 330 | 399 | 822 | 776 | 596 |
| 796 | 368 | 263 | 308 | 810 | 770 | 566 |
| 777 | 331 | 214 | 267 | 797 | 743 | 542 |
| 773 | 265 | 189 | 212 | 779 | 719 | 532 |
| 768 | 221 | 158 | 187 | 742 | 634 | 475 |
| 741 | 190 | | 161 | 716 | 619 | 447 |
| 717 | 158 | | | 703 | 598 | 406 |
| 644 | | | | 663 | 572 | 381 |
| 632 | | | | 625 | 537 | 369 |
| 592 | | | | 597 | 522 | 343 |
| 559 | | | | 574 | 471 | 330 |
| 534 | | | | 541 | 444 | 268 |
| 465 | | | | 510 | 430 | 218 |
| 447 | | | | 469 | 352 | 190 |
| 265 | | | | 443 | 308 | 173 |
| 233 | | | | 370 | 266 | 157 |
| 296 | | | | 344 | 258 | |
| 266 | | | | 272 | 229 | |
| 246 | | | | 217 | 191 | |
| 215 | | | | 184 | 154 | |
| 189 | | | | 170 | 118 | |
| 160 | | | | 152 | | |

| sesqui- | mono- | mono- | THF/ | |
|---|---|---|---|---|
| dioxane | DMF | NMP | water | modification |
| solvate | solvate | solvate | form | II |
| 3067 | 3140 | 3072 | 3063 | 3397 |
| 3021 | 3081 | 3038 | 3027 | 3143 |
| 2964 | 3060 | 2935 | 2955 | 3095 |
| 2939 | 3028 | 2837 | 2602 | 3079 |
| 2890 | 3006 | 1734 | 1707 | 3056 |
| 2859 | 2955 | 1657 | 1618 | 3025 |
| 2721 | 2841 | 1636 | 1600 | 3006 |
| 1719 | 2601 | 1619 | 1576 | 2956 |
| 1615 | 1727 | 1601 | 1509 | 2843 |
| 1599 | 1664 | 1572 | 1447 | 1728 |
| 1570 | 1612 | 1508 | 1421 | 1641 |
| 1504 | 1602 | 1488 | 1381 | 1619 |
| 1481 | 1573 | 1445 | 1353 | 1567 |
| 1446 | 1509 | 1420 | 1323 | 1567 |
| 1419 | 1493 | 1385 | 1308 | 1503 |
| 1389 | 1484 | 1371 | 1277 | 1478 |
| 1372 | 1441 | 1325 | 1250 | 1442 |
| 1328 | 1423 | 1305 | 1231 | 1434 |
| 1307 | 1389 | 1292 | 1178 | 1417 |
| 1290 | 1372 | 1254 | 1156 | 1385 |
| 1274 | 1355 | 1224 | 1143 | 1372 |
| 1255 | 1322 | 184 | 1114 | 1344 |
| 1228 | 1306 | 1172 | 1058 | 1322 |
| 1217 | 1275 | 1153 | 1035 | 1308 |
| 1190 | 1248 | 1111 | 1004 | 1288 |
| 1172 | 1228 | 1094 | 964 | 1277 |
| 1158 | 1180 | 1065 | 911 | 1244 |
| 1142 | 1142 | 1029 | 823 | 1232 |
| 1108 | 1115 | 963 | 810 | 1172 |
| 1096 | 1096 | 929 | 797 | 1144 |
| 1037 | 1056 | 912 | 774 | 1115 |
| 1028 | 1034 | 828 | 742 | 1059 |
| 1015 | 1003 | 811 | 717 | 1032 |
| 953 | 968 | 797 | 592 | 964 |
| 854 | 942 | 772 | 559 | 906 |
| 834 | 907 | 741 | 536 | 820 |
| 818 | 869 | 706 | 470 | 800 |
| 794 | 825 | 664 | 447 | 772 |
| 778 | 810 | 644 | 410 | 740 |
| 773 | 781 | 620 | 333 | 719 |
| 740 | 773 | 596 | 266 | 646 |
| 715 | 765 | 576 | 254 | 629 |
| 705 | 745 | 566 | 216 | 1308 |
| 560 | 717 | 559 | 189 | 1288 |
| 544 | 665 | 539 | 153 | 1277 |
| 529 | 635 | 513 | | 1244 |
| 595 | 620 | 477 | | 1232 |
| 560 | 592 | 443 | | 1172 |
| 536 | 569 | 403 | | 1144 |
| 512 | 536 | 359 | | 1115 |
| 491 | 523 | 331 | | 1059 |
| 477 | 470 | 308 | | 1032 |
| 442 | 450 | 283 | | 964 |
| 367 | 410 | 265 | | 906 |
| 342 | 357 | 222 | | 820 |
| 287 | 313 | 183 | | 800 |
| 264 | 268 | 172 | | 772 |
| 231 | 256 | 155 | | 740 |
| 215 | 240 | | | 719 |
| 188 | 229 | | | 646 |
| 161 | 221 | | | 629 |
| | 190 | | | 588 |
| | 154 | | | 556 |
| | 113 | | | 538 |
| | | | | 526 |
| | | | | 467 |
| | | | | 436 |
| | | | | 349 |
| | | | | 316 |
| | | | | 268 |

TABLE 5
FIR spectroscopy
Wave number [cm⁻¹]

| | | | | semi- | monoiso- | di- |
|---|---|---|---|---|---|---|
| modification | meso- | semi- | mono- | ethanol | propanol | DMSO |
| I | morphic | hydrate | hydrate | solvate | solvate | solvate |
| 487 | 495 | 464 | 479 | 494 | 494 | 475 |
| 466 | 485 | 447 | 456 | 489 | 470 | 451 |
| 451 | 469 | 405 | 437 | 482 | 447 | 439 |
| 430 | 447 | 343 | 368 | 475 | 427 | 404 |
| 407 | 430 | 292 | 333 | 469 | 397 | 381 |
| 365 | 406 | 262 | 311 | 457 | 377 | 344 |
| 343 | 368 | 243 | 232 | 451 | 347 | 329 |
| 325 | 331 | 188 | 186 | 441 | 328 | 319 |
| 318 | 289 | 162 | 171 | 369 | 307 | 283 |
| 291 | 262 | 120 | | 339 | 287 | 266 |
| 262 | 236 | | | 316 | 261 | 223 |
| 237 | 188 | | | 287 | 249 | 172 |
| 218 | 160 | | | 270 | 236 | 149 |
| 188 | 108 | | | 254 | 227 | 145 |
| 160 | 92 | | | 218 | 215 | 141 |
| | | | | 171 | 171 | 116 |
| | | | | 157 | 160 | 105 |
| | | | | 111 | 140 | 89 |
| | | | | | 128 | 85 |
| | | | | | 109 | |
| | | | | | 104 | |

| sesqui- | mono- | mono- | THF/ | |
|---|---|---|---|---|
| dioxane | DMF | NMP | water | modification |
| solvate | solvate | solvate | form | II |
| 494 | 494 | 488 | 491 | 461 |
| 476 | 488 | 477 | 482 | 447 |
| 462 | 484 | 448 | 470 | 435 |
| 437 | 470 | 435 | 449 | 426 |
| 433 | 452 | 411 | 430 | 405 |
| 402 | 437 | 369 | 404 | 362 |
| 366 | 428 | 355 | 377 | 346 |
| 340 | 408 | 315 | 327 | 324 |
| 324 | 378 | 307 | 288 | 304 |
| 304 | 363 | 279 | 262 | 246 |
| 289 | 354 | 255 | 248 | 240 |
| 275 | 325 | 229 | 233 | 212 |
| 254 | 311 | 215 | 186 | 194 |
| 235 | 290 | 180 | 161 | 167 |
| 227 | 264 | 169 | 112 | 146 |
| 186 | 249 | 155 | | 104 |
| 164 | 236 | 119 | | 96 |
| 89 | 214 | 104 | | 92 |
| | 185 | 93 | | 84 |
| | 163 | | | |
| | 109 | | | |

TABLE 6
NIR spectroscopy
Wave number [cm⁻¹]

| | | | | semi- | monoiso- | di- |
|---|---|---|---|---|---|---|
| modification | meso- | semi- | mono- | ethanol | propanol | DMSO |
| I | morphic | hydrate | hydrate | solvate | solvate | solvate |
| 9793 | 8789 | 8763 | 8720 | 8831 | 8801 | 8782 |
| 8779 | 8408 | 7150 | 8484 | 8442 | 8456 | 8502 |
| 7828 | 7107 | 6843 | 7863 | 7840 | 7851 | 7363 |
| 6834 | 6846 | 6643 | 7120 | 6851 | 7114 | 7209 |
| 6724 | 6636 | 6049 | 6877 | 6717 | 6855 | 6804 |
| 6631 | 5977 | 5986 | 6659 | 6631 | 6683 | 6562 |
| 6328 | 5244 | 5853 | 6536 | 6335 | 5936 | 5976 |
| 6059 | 5057 | 5601 | 6015 | 6017 | 5893 | 5936 |
| 5984 | 4984 | 5275 | 5965 | 5980 | 5781 | 5772 |
| 5846 | 4802 | 5099 | 5775 | 5888 | 5651 | 5687 |
| 5593 | 4660 | 5057 | 5741 | 5822 | 5263 | 5601 |
| 5095 | 4432 | 5033 | 5263 | 5710 | 5107 | 5494 |
| 5058 | 4149 | 4965 | 5125 | 5629 | 4973 | 5317 |
| 4965 | 4056 | 4873 | 5064 | 5094 | 4920 | 5080 |
| 4916 | | 4817 | 5006 | 4965 | 4736 | 4979 |
| 4865 | | 4658 | 4804 | 4866 | 4661 | 4792 |
| 4808 | | 4610 | 4669 | 4808 | 4539 | 4633 |
| 4646 | | 4493 | 4498 | 4714 | 4490 | 4398 |
| 4595 | | 4425 | 4447 | 4633 | 4444 | 4283 |
| 4531 | | 4314 | 4357 | 4541 | 4399 | 4210 |
| 4485 | | 4210 | 4198 | 4485 | 4320 | 4151 |
| 4419 | | 4168 | 4163 | 4446 | 4219 | 4085 |
| 4348 | | 4147 | 4071 | 4403 | 4139 | 4016 |
| 4268 | | 4048 | | 4368 | 4080 | |
| 4199 | | | | 4330 | | |
| 4062 | | | | 4219 | | |
| | | | | 4163 | | |
| | | | | 4105 | | |
| | | | | 4072 | | |

| sesqui- | mono- | mono- | THF/ | |
|---|---|---|---|---|
| dioxane | DMF | NMP | water | modification |
| solvate | solvate | solvate | form | II |
| 9856 | 8801 | 8774 | 8797 | 9786 |
| 8795 | 8455 | 8462 | 8444 | 8798 |
| 8460 | 7852 | 7853 | 7037 | 8534 |
| 7257 | 6860 | 6862 | 5846 | 8450 |
| 6870 | 6746 | 6781 | 5633 | 8152 |
| 6736 | 6628 | 6656 | 5966 | 7866 |
| 6637 | 6344 | 6431 | 5229 | 6949 |
| 6527 | 6145 | 6030 | 5097 | 6842 |
| 6315 | 5958 | 5948 | 5063 | 6784 |
| 6012 | 5778 | 5768 | 4969 | 6666 |
| 5932 | 5651 | 5111 | 4862 | 6357 |
| 5862 | 5241 | 5069 | 4805 | 6044 |
| 5775 | 5099 | 4978 | 4651 | 5971 |
| 5584 | 5062 | 4797 | 4593 | 5874 |
| 5376 | 4974 | 4750 | 4536 | 5811 |
| 5046 | 4907 | 4664 | 4489 | 5625 |
| 4978 | 4738 | 4551 | 4433 | 5429 |
| 4919 | 4661 | 4494 | 4350 | 5231 |
| 4850 | 4630 | 4427 | 4199 | 5107 |
| 4782 | 4540 | 4374 | 4166 | 5067 |
| 4644 | 4488 | 4297 | 4061 | 5004 |
| 4528 | 4435 | 4216 | | 4965 |
| 4483 | 4393 | 4177 | | 4891 |
| 4436 | 4278 | 4158 | | 4836 |
| 4399 | 4231 | 4097 | | 4805 |
| 4350 | 4194 | 4067 | | 4732 |
| 4273 | 4167 | | | 4659 |
| 5209 | 4139 | | | 4553 |
| 4159 | 4080 | | | 4503 |
| 4085 | 4020 | | | 4481 |
| | | | | 4443 |
| | | | | 4402 |
| | | | | 4367 |
| | | | | 4329 |
| | | | | 4262 |
| | | | | 4164 |
| | | | | 4120 |
| | | | | 4057 |
| | | | | 4037 |

TABLE 7
¹³C Solid state NMR spectroscopy
ppm

| | | | | semi- | monoiso- | di- |
|---|---|---|---|---|---|---|
| modification | meso- | semi- | mono- | ethanol | propanol | DMSO |
| I | morphic | hydrate | hydrate | solvate | solvate | solvate |
| 52 | 22 | 52 | 53 | 21 | 23 | 41 |
| 95 | 26 | 95 | 93 | 54 | 32 | 45 |
| 116 | 31 | 115 | 116 | 59 | 42 | 52 |
| 123 | 35 | 116 | 127 | 94 | 52 | 94 |
| 126 | 41 | 122 | 131 | 117 | 64 | 116 |
| 128 | 52 | 124 | 134 | 125 | 94 | 119 |
| 130 | 96 | 125 | 142 | 129 | 113 | 125 |
| 133 | 115 | 127 | 147 | 131 | 116 | 127 |
| 138 | 124 | 130 | 150 | 143 | 124 | 132 |
| 141 | 128 | 131 | 159 | 146 | 126 | 135 |
| 149 | 132 | 134 | | 151 | 132 | 143 |
| 150 | 141 | 136 | | 158 | 142 | 152 |
| 158 | 149 | 140 | | 161 | 148 | 156 |
| 161 | 158 | 142 | | | 158 | 158 |
| | 161 | 149 | | | 160 | 161 |
| | | 157 | | | 163 | |
| | | 161 | | | | |

| sesqui- | mono- | mono- | THF/ | |
|---|---|---|---|---|
| dioxane | DMF | NMP | water | modification |
| solvate | solvate | solvate | form | II |
| 53 | 29 | 29 | 31 | 53 |
| 68 | 37 | 51 | 52 | 94 |
| 97 | 53 | 94 | 95 | 116 |
| 116 | 96 | 114 | 113 | 122 |
| 126 | 113 | 116 | 116 | 124 |
| 130 | 117 | 120 | 123 | 130 |
| 134 | 126 | 126 | 128 | 131 |
| 142 | 128 | 130 | 132 | 135 |
| 147 | 132 | 131 | 142 | 142 |
| 149 | 142 | 140 | 149 | 147 |
| 155 | 150 | 150 | 158 | 149 |
| 157 | 158 | 158 | 161 | 150 |
| 160 | 161 | 162 | | 154 |
| | 164 | 177 | | 158 |
| | | | | 161 |

TABLE 8
Crystal structure data

| | monoisopropanol | semi-ethanol | di-DMSO | sesquidioxane | mono-DMF |
|---|---|---|---|---|---|
| | solvate | solvate | solvate | solvate | solvate |
| temperature [K] | 100 | 100 | 100 | 100 | 100 |
| crystal system | monocline | monocline | monocline | tricline | monocline |
| space group | C2/c | P2(1)/c | P2(1)/c | P-1 | C2/c |
| molecules per unit | 8 | 4 | 4 | 2 | 8 |
| cell | | | | | |
| length of axis a [Å] | 18.2447(19) | 14.8608(13) | 12.76500(10) | 10.5316(5) | 18.17650(10) |
| length of axis b [Å] | 13.0500(13) | 18.1393(11) | 11.73540(10) | 11.8238(6) | 13.20440(10) |
| length of axis c [Å] | 19.3712(16) | 7.2437(4) | 17.98500(10) | 11.8614(5) | 19.02370(10) |
| α [°] | 90 | 90 | 90 | 69.890(4) | 90 |
| β [°] | 99.270(8) | 91.494(6) | 92.1020(10) | 86.794(4) | 97.2380(10) |
| γ [°] | 90 | 90 | 90 | 66.146(5) | 90 |
| calculated density | 1.367 | 1.468 | 1.363 | 1.422 | 1412 |
| [g cm⁻³] | | | | | |

FIG. 1: DSC and TGA thermograms of methyl {4,6-diamino-2-[1-(2-fluorobenzyl)-1H-pyrazolo[3,4-b]pyridin-3-yl]pyrimidin-5-yl}carbamate

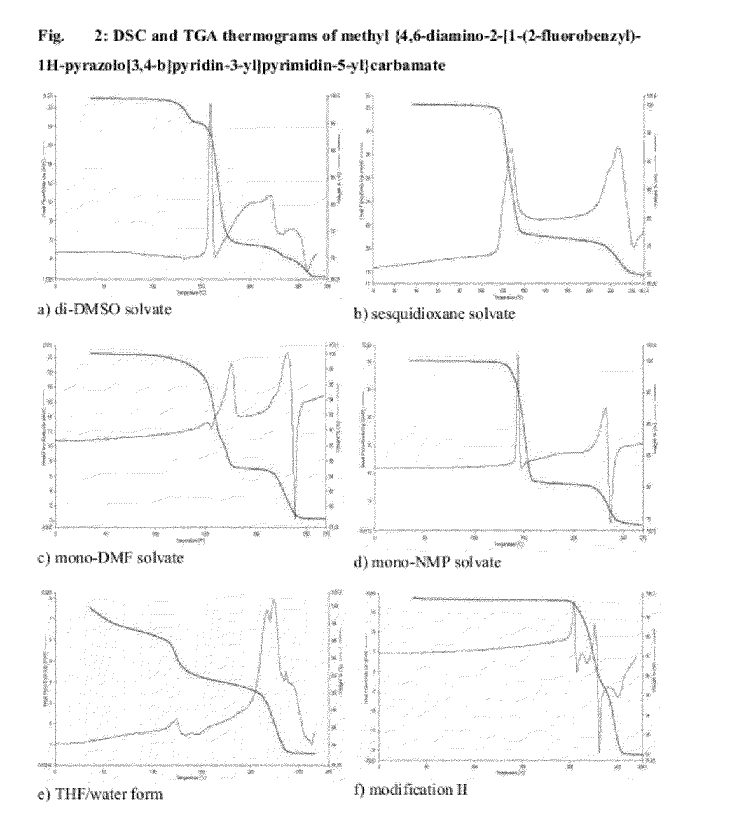

FIG. 2: DSC and TGA thermograms of methyl {4,6-diamino-2-[1-(2-fluorobenzyl)-1H-pyrazolo[3,4-b]pyridin-3-yl]pyrimidin-5-yl}carbamate

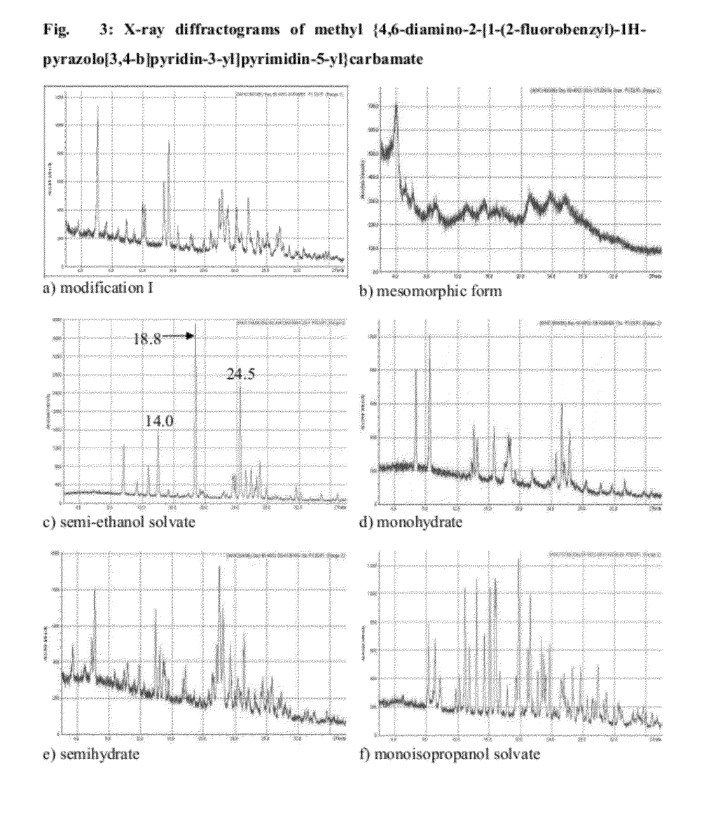

FIG. 3: X-ray diffractograms of methyl {4,6-diamino-2-[1-(2-fluorobenzyl)-1H-pyrazolo[3,4-b]pyridin-3-yl]pyrimidin-5-yl}carbamate

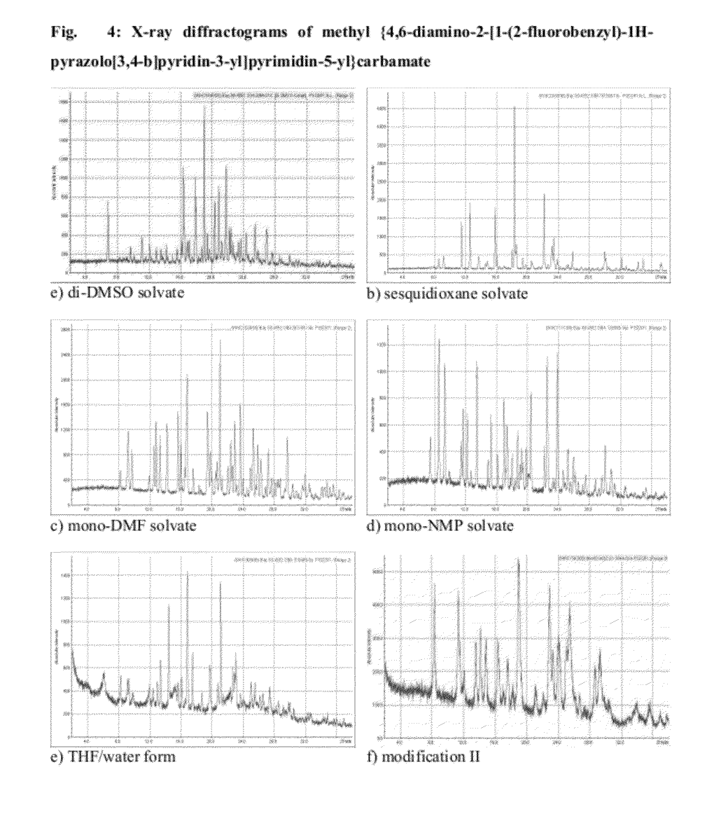

FIG. 4: X-ray diffractograms of methyl {4,6-diamino-2-[1-(2-fluorobenzyl)-1H-pyrazolo[3,4-b]pyridin-3-yl]pyrimidin-5-yl}carbamate

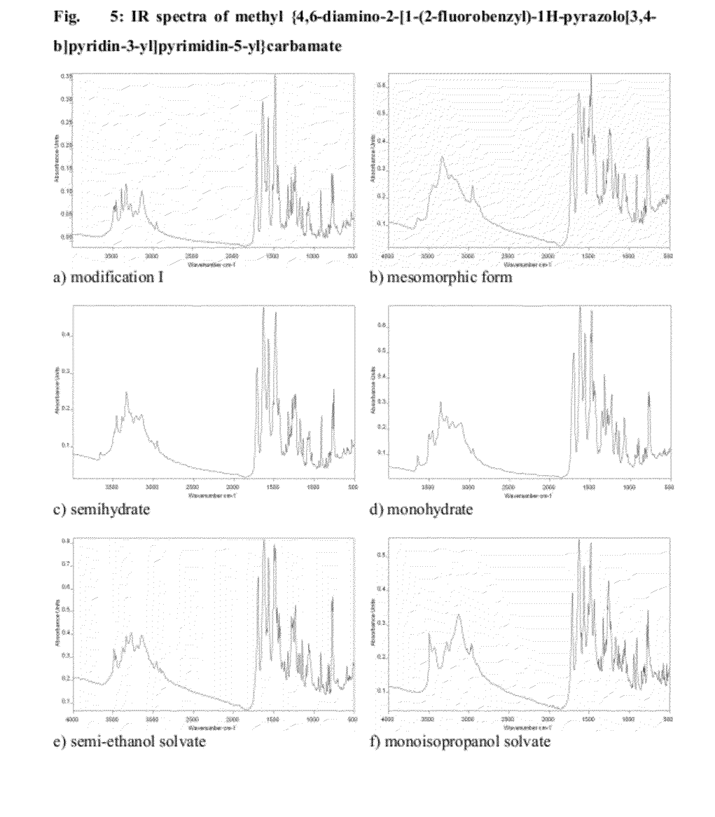

FIG. 5: IR spectra of methyl {4,6-diamino-2-[1-(2-fluorobenzyl)-1H-pyrazolo[3,4-b]pyridin-3-yl]pyrimidin-5-yl}carbamate

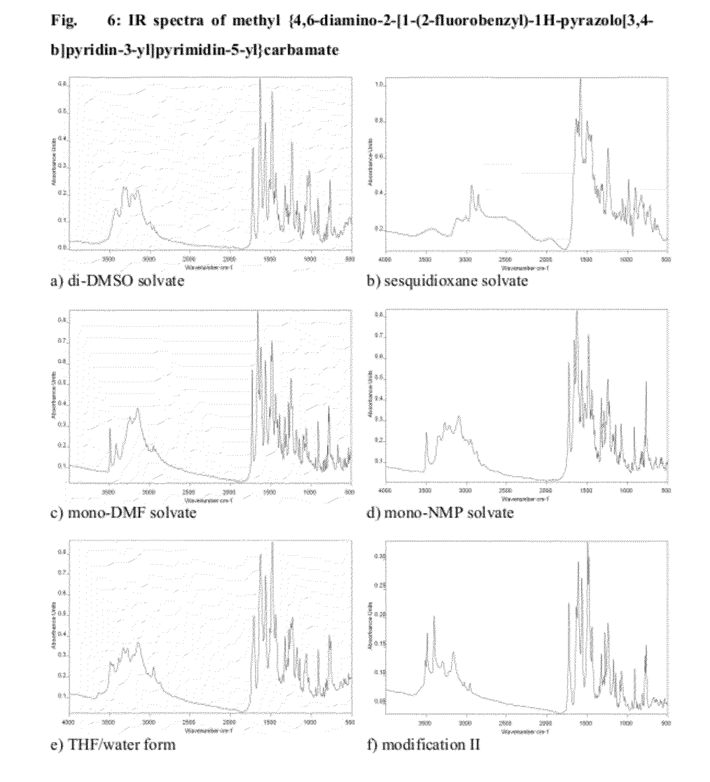

FIG. 6: IR spectra of methyl {4,6-diamino-2-[1-(2-fluorobenzyl)-1H-pyrazolo[3,4-b]pyridin-3-yl]pyrimidin-5-yl}carbamate

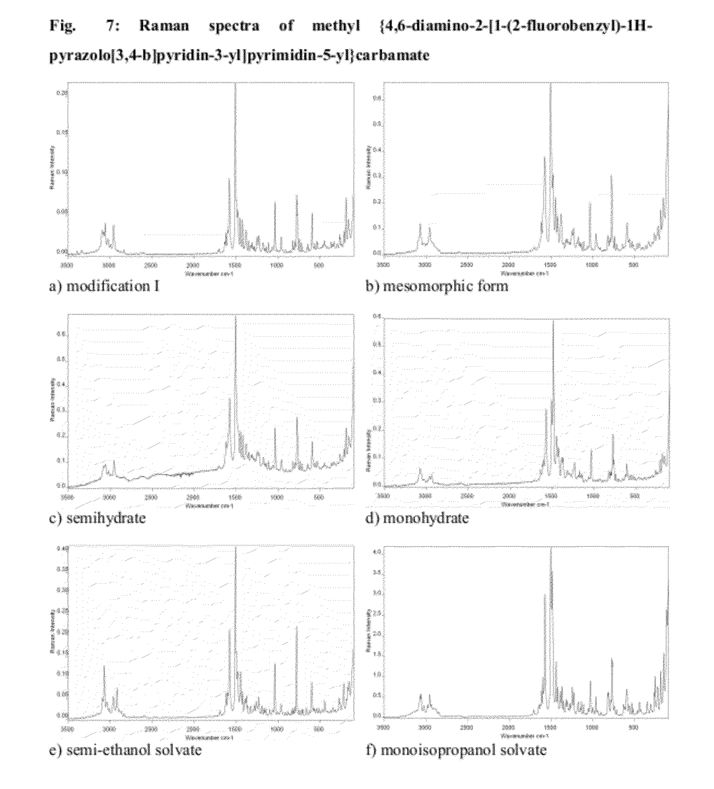

FIG. 7: Raman spectra of methyl {4,6-diamino-2-[1-(2-fluorobenzyl)-1H-pyrazolo[3,4-b]pyridin-3-yl]pyrimidin-5-yl}carbamate

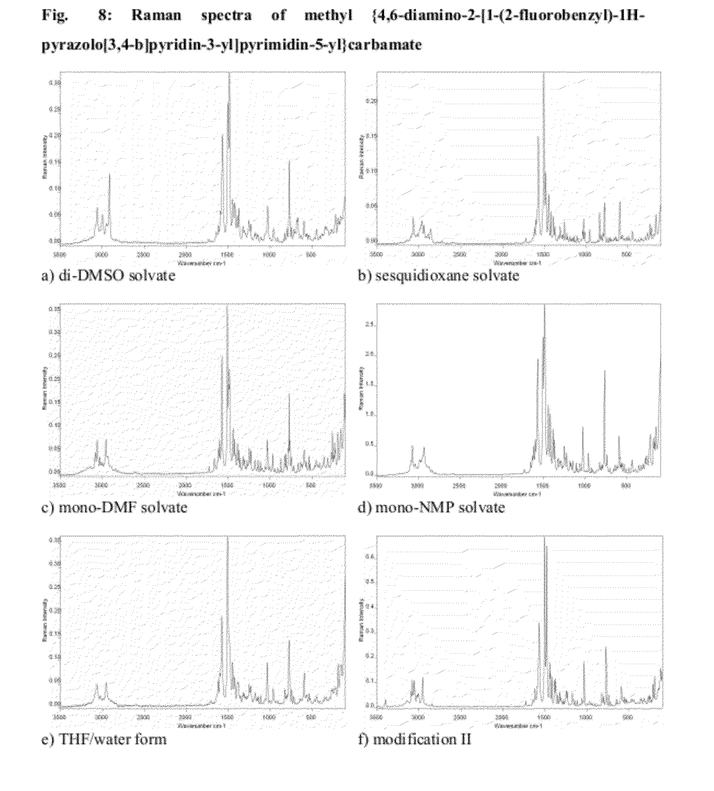

FIG. 8: Raman spectra of methyl {4,6-diamino-2-[1-(2-fluorobenzyl)-1H-pyrazolo[3,4-b]pyridin-3-yl]pyrimidin-5-yl}carbamate

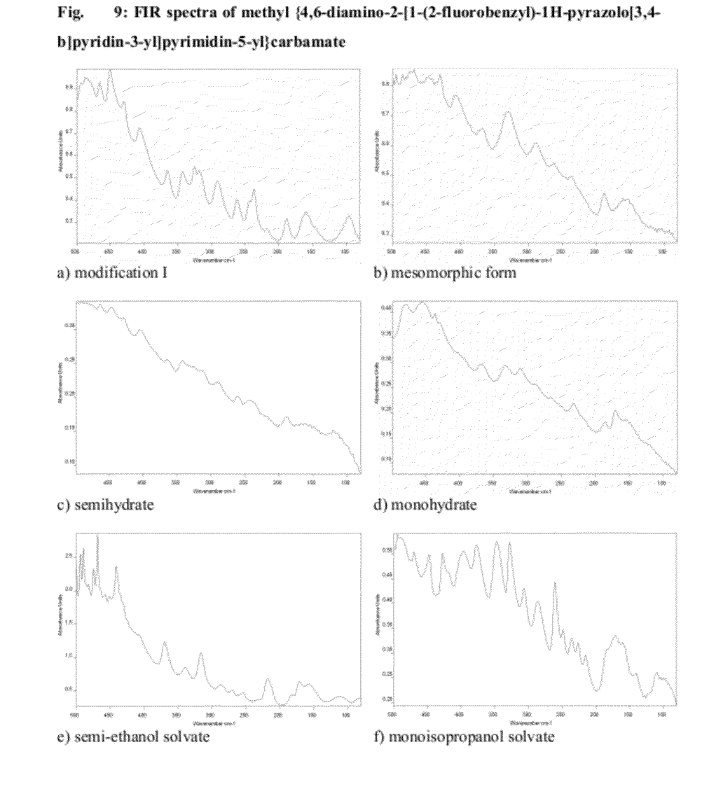

FIG. 9: FIR spectra of methyl {4,6-diamino-2-[1-(2-fluorobenzyl)-1H-pyrazolo[3,4-b]pyridin-3-yl]pyrimidin-5-yl}carbamate

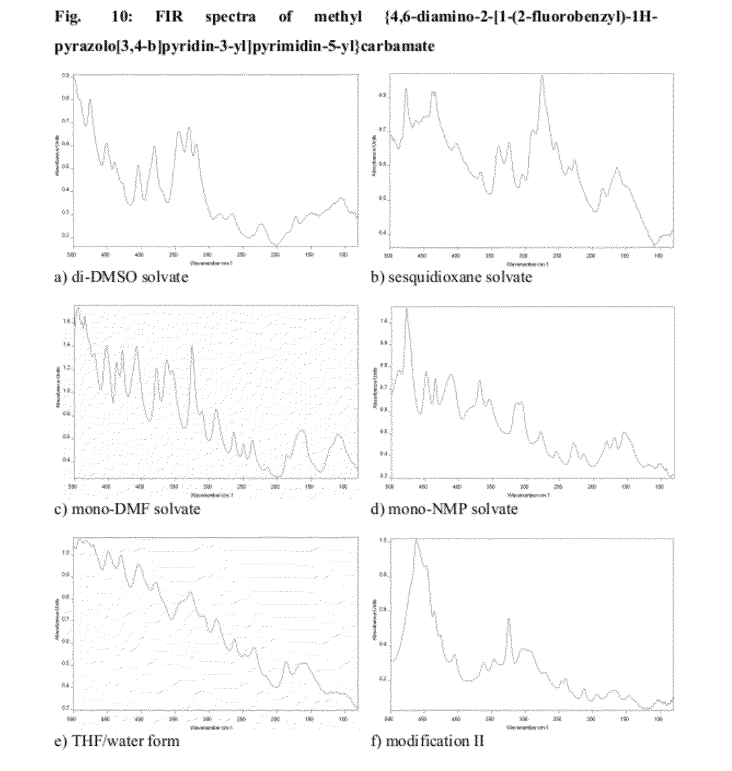

FIG. 10: FIR spectra of methyl {4,6-diamino-2-[1-(2-fluorobenzyl)-1H-pyrazolo[3,4-b]pyridin-3-yl]pyrimidin-5-yl}carbamate

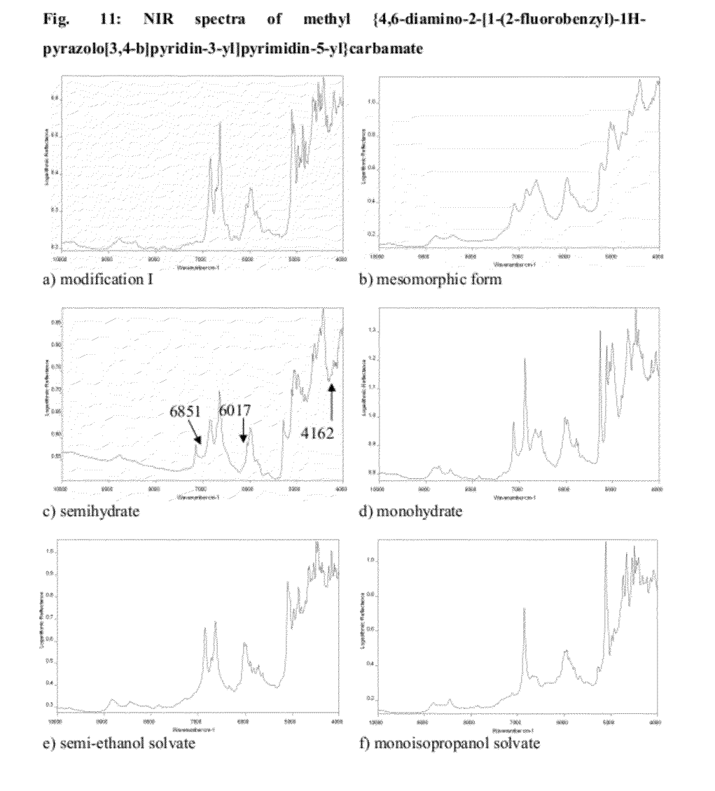

FIG. 11: NIR spectra of methyl {4,6-diamino-2-[1-(2-fluorobenzyl)-1H-pyrazolo[3,4-b]pyridin-3-yl]pyrimidin-5-yl}carbamate

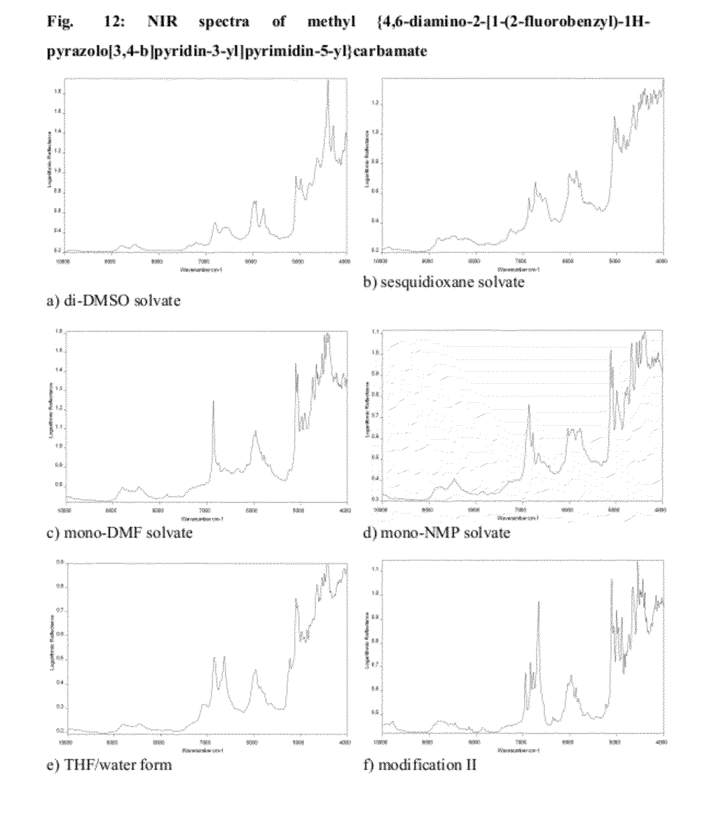

FIG. 12: NIR spectra of methyl {4,6-diamino-2-[1-(2-fluorobenzyl)-1H-pyrazolo[3,4-b]pyridin-3-yl]pyrimidin-5-yl}carbamate

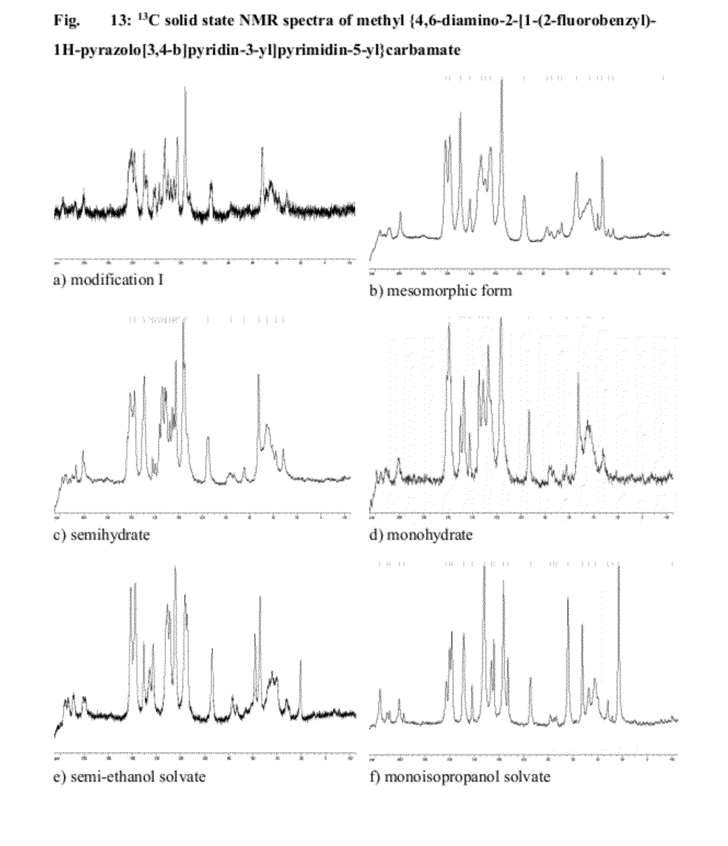

FIG. 13: ¹³C solid state NMR spectra of methyl {4,6-diamino-2-[1-(2-fluorobenzyl)-1H-pyrazolo[3,4-b]pyridin-3-yl]pyrimidin-5-yl}carbamate

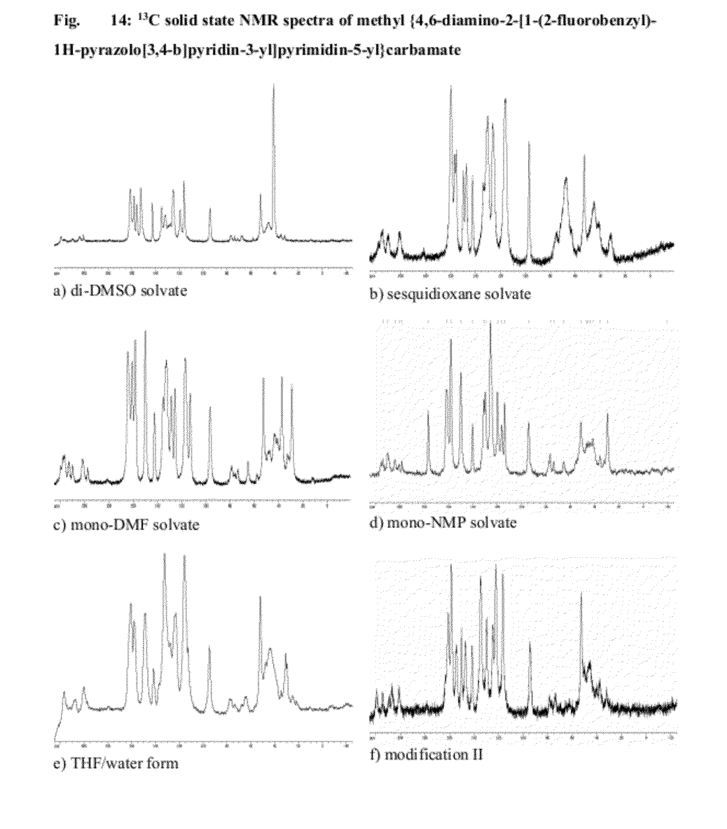

FIG. 14: ¹³C solid state NMR spectra of methyl {4,6-diamino-2-[1-(2-fluorobenzyl)-1H-pyrazolo[3,4-b]pyridin-3-yl]pyrimidin-5-yl}carbamate

FIG. 15: Calculated X-ray diffractogram and molecular geometry of the semi-ethanol solvate of the formula (Ia)

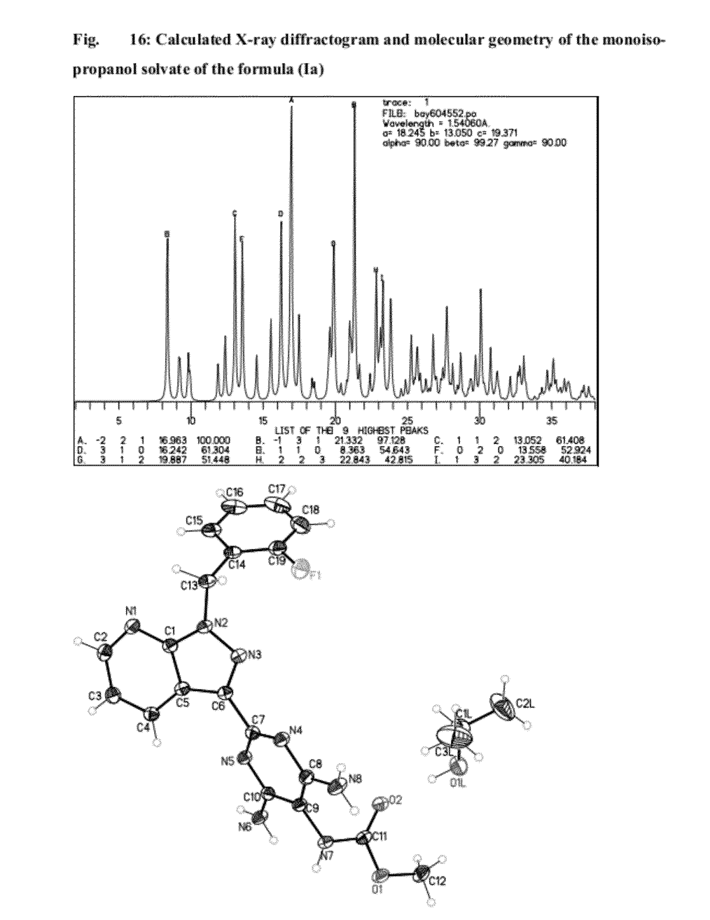

FIG. 16: Calculated X-ray diffractogram and molecular geometry of the monoisopropanol solvate of the formula (Ia)

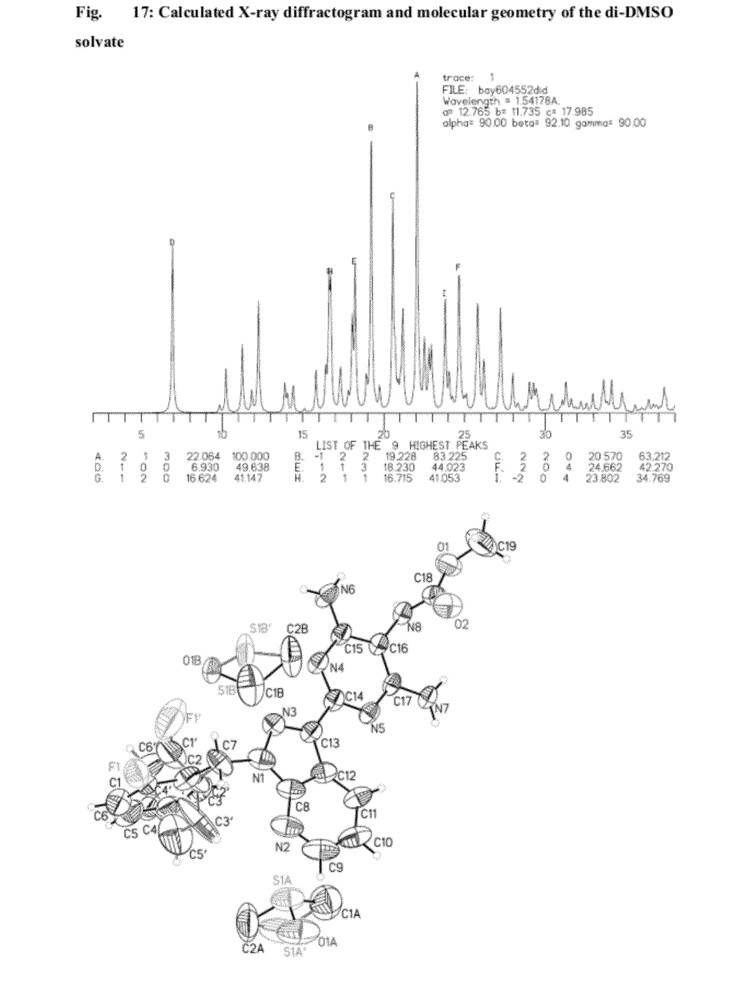

FIG. 17: Calculated X-ray diffractogram and molecular geometry of the di-DMSO solvate

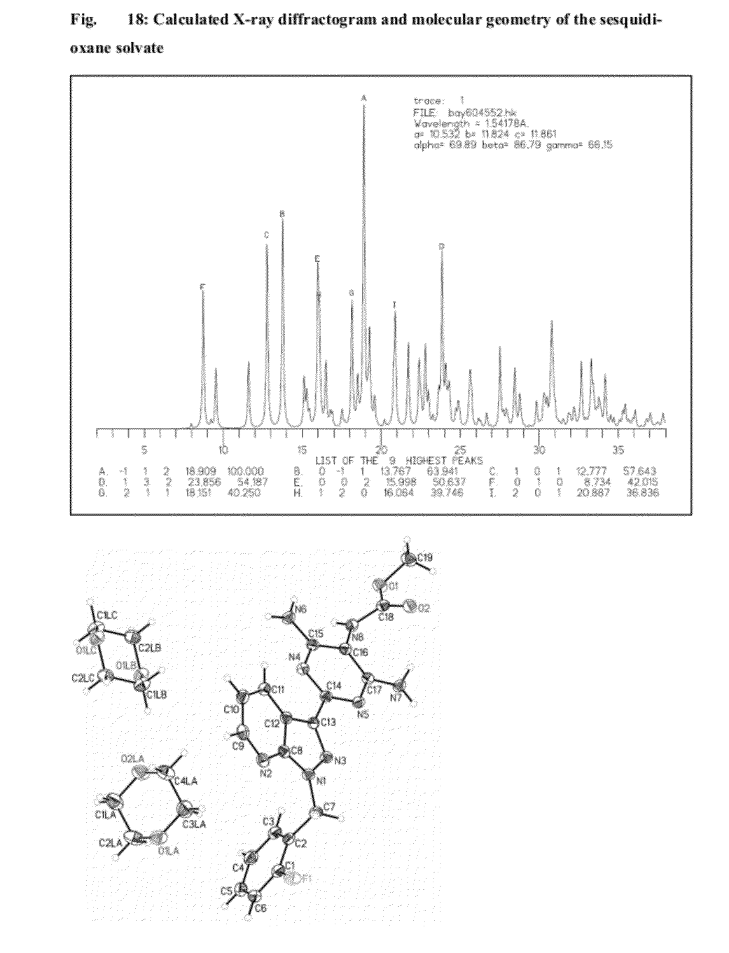

FIG. 18: Calculated X-ray diffractogram and molecular geometry of the sesquidioxane solvate

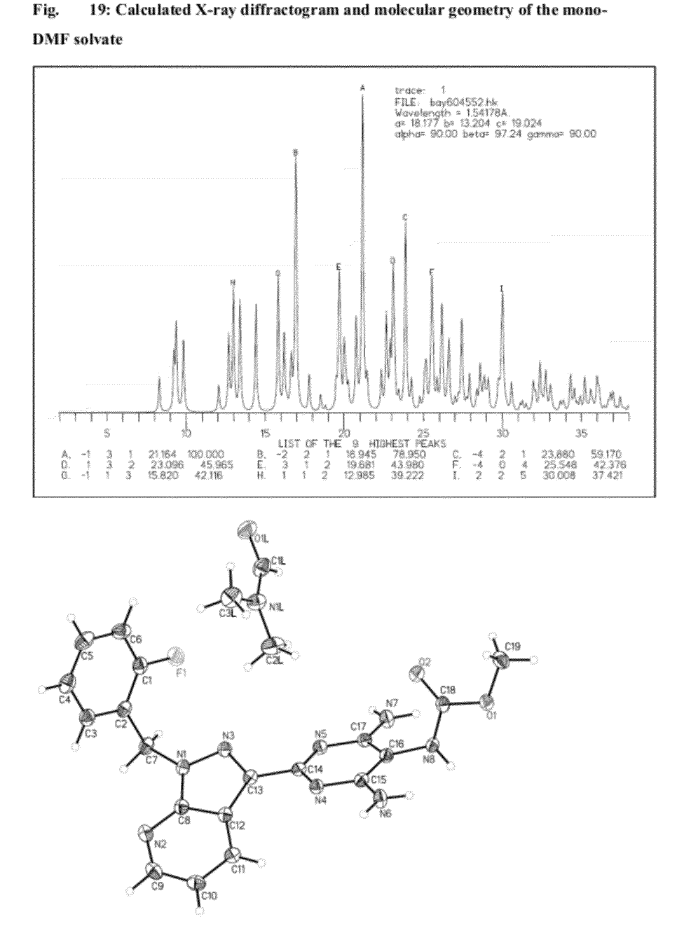

FIG. 19: Calculated X-ray diffractogram and molecular geometry of the mono-DMF solvate

1. Methyl {4,6-diamino-2-[1-(2-fluorobenzyl)-1H-pyrazolo[3,4-b]pyridin-3-yl]pyrimidin-5-yl}carbamate of the formula (I) as semi-ethanol solvate of the formula (Ia)

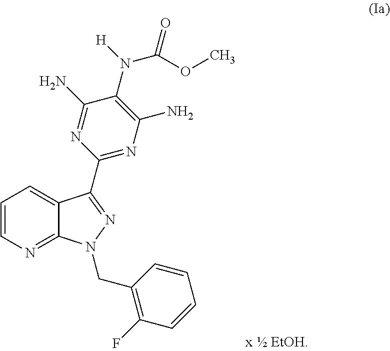

2. The compound of claim 1, characterized in that the X-ray diffractogram of the compound has a peak maximum of the 2 theta angle at 18.8.

3. The compound of claim 1, characterized in that the X-ray diffractogram of the compound has peak maxima of the 2 theta angle at 14.0, 18.8 and 24.5.

4. The compound of claim 1, characterized in that the NIR spectrum of the compound has peak maxima at 6851 cm⁻¹, 6017 cm⁻¹ and 4163 cm⁻¹.

5. (canceled)

6. A pharmaceutical composition comprising a compound a of claim 1 and no major fractions of any other form of the compound of the formula (Ia).

7. A pharmaceutical composition comprising a compounded of claim 1 in an amount of more than 90 percent by weight based on the total amount of the compound of the formula (Ia) present.

8. A process for preparing the compound of claim 1 comprising: suspending the compound of the formula (I) 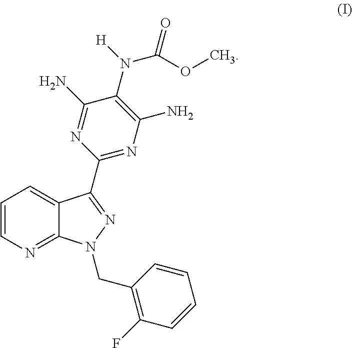
in a mesomorphic form in an ethanol-comprising solvent, and
stirring or shaking at a temperature of from 10° C. to the reflux temperature of the solvent until quantitative conversion into the semi-ethanol solvate of the formula (Ia), as defined in claim 1 has been achieved.

9. (canceled)

10. A method for treating a cardiovascular disorders by administering an effective amount of a compound of claim 1 to a subject in need thereof.

11. The method of claim 10, wherein the cardiovascular disorder is selected from the group consisting of hypertension and heart failure, stable and unstable angina pectoris, a peripheral cardiac vascular disorder, and arrhythmia.
